SEQUENCE LISTING (70 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 174 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTS

SEQ ID NO: 3 - protein, 156 aa, Homo sapiens [sequence omitted]

SEQ ID NO: 4 - protein, 112 aa, Artificial sequence
  Variant ligand binding domain of human TREM2
  VARIANT (78)..(78): X is a basic amino acid, preferably K or R
HNTTVFQGVAGQSLQVSCPYDSMKHWGRRKAWCRQLGEKGPCQRVVSTHNLWLLSFLRRWNGSTAITDDTLGGTLTIXLRNLQPHDAGLYQCQSLHGSEADTLRKVLVEVLA

SEQ ID NO: 5 - protein, 120 aa, Homo sapiens
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDA

SEQ ID NO: 6 - protein, 120 aa, Artificial sequence
  Variant ligand binding domain of TREM 2
  VARIANT (78)..(78): X is a basic amino acid, preferably K or R
HNTTVFQGVAGQSLQVSCPYDSMKHWGRRKAWCRQLGEKGPCQRVVSTHNLWLLSFLRRWNGSTAITDDTLGGTLTIXLRNLQPHDAGLYQCQSLHGSEADTLRKVLVEVLADPLDHRDA

SEQ ID NO: 7 - protein, 156 aa, Artificial sequence
  Variant ligand binding domain of TREM 2
  VARIANT (139)..(140): X at position 139 is not H and/or X at position 140 is not S
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEXXISRSLLEGEIPFPPTS

SEQ ID NO: 8 - protein, 156 aa, Artificial sequence
  Variant ligand binding domain of TREM 2
  VARIANT (78)..(78): X is a basic amino acid, preferably K or R
  VARIANT (139)..(140): X at position 139 is not H and/or X at position 140 is not S
HNTTVFQGVAGQSLQVSCPYDSMKHWGRRKAWCRQLGEKGPCQRVVSTHNLWLLSFLRRWNGSTAITDDTLGGTLTIXLRNLQPHDAGLYQCQSLHGSEADTLRKVLVEVLADPLDHRDAGDLWFPGESESFEDAHVEXXISRSLLEGEIPFPPTS

SEQ ID NO: 9 - protein, 21 aa, Homo sapiens
MALPVTALLLPLALLLHAARP

SEQ ID NO: 10 - protein, 18 aa, Homo sapiens
MEPLRLLILLFVTELSGA

SEQ ID NO: 11 - protein, 43 aa, Homo sapiens
IEVEQKLISEEDLLDNEKSNGTIIHVKGKHLCPSPLFPGPSKP

SEQ ID NO: 12 - protein, 27 aa, Homo sapiens
FWVLVVVGGVLACYSLLVTVAFIIFWV

SEQ ID NO: 13 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 15 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 16 - protein, 230 aa, Homo sapiens
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 17 - protein, 569 aa, Homo sapiens
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 18 - protein, 437 aa, Homo sapiens
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 19 - protein, 189 aa, Homo sapiens
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 20 - protein, 91 aa, Homo sapiens
KKRIKPIVWPSLPDHKKTLEHLCKKPRKNLNVSFNPESFLDCQIHRVDDIQARDEVEGFLQDTFPQQPILTSLGSNQEEAYVTMSSFYQNQ

SEQ ID NO: 21 - protein, 57 aa, Homo sapiens
KVLKCNTPDPSKFFSQLSSEHGGDVQKWLSSPFPSSSFSPGGLAPEISPLEVLERDK

SEQ ID NO: 22 - protein, 45 aa, Homo sapiens
NPWFQRAKMPRALDFSGHTHPVATFQPSRPESVNDLFLCPQKELT

SEQ ID NO: 23 - protein, 42 aa, Homo sapiens
GYICLRNSLPKVLNFHNFLAWPFPNLPPLEAMDMVEVIYINR

SEQ ID NO: 24 - protein, 42 aa, Homo sapiens
PLKEKSIILPKSLISVVRSATLETKPESKYVSLITSYQPFSL

SEQ ID NO: 25 - protein, 43 aa, Homo sapiens
RRRKKLPSVLLFKKPSPFIFISQRPSPETQDTIHPLDEEAFLK

SEQ ID NO: 26 - protein, 45 aa, Homo sapiens
YIHVGKEKHPANLILIYGNEFDKRFFVPAEKIVINFITLNISDDS

SEQ ID NO: 27 - protein, 41 aa, Homo sapiens
RYVTKPPAPPNSLNVQRVLTFQPLRFIQEHVLIPVFDLSGP

SEQ ID NO: 28 - protein, 43 aa, Homo sapiens
NYVFFPSLKPSSSIDEYFSEQPLKNLLLSTSEEQIEKCFIIEN

SEQ ID NO: 29 - protein, 49 aa, Homo sapiens
YWFHTPPSIPLQIEEYLKDPTQPILEALDKDSSPKDDVWDSVSIISFPE

SEQ ID NO: 30 - protein, 51 aa, Homo sapiens
YAFSPRNSLPQHLKEFLGHPHHNTLLFFSFPLSDENDVFDKLSVIAEDSES

SEQ ID NO: 31 - protein, 41 aa, Homo sapiens
RSKRSRLLHSDYMNMTPRRPGPTRKHYQPYAPPRDFAAYRS

SEQ ID NO: 32 - protein, 364 aa, Artificial sequence
  TREM2 LBD Chimeric receptor
MALPVTALLLPLALLLHAARPHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 364 aa, Artificial sequence
  TREM2 mutant LBD chimeric receptor
MALPVTALLLPLALLLHAARPHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1092 bp, Artificial sequence
  TREM2 LBD chimeric receptor
atggccctgcccgtgaccgccctgctgctgcccctggccctgctgctgcacgccgcccggcctcacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - DNA, 1092 bp, Artificial sequence
  TREM2 mutant LBD chimeric receptor
atggccctgcccgtgaccgccctgctgctgcccctggccctgctgctgcacgccgcccggcctcacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattaagctgcggaatctacaaccccatgatgcgggtctctaccagtgccagagcctccatggcagtgaggctgacaccctcaggaaggtcctggtggaggtgctggcagaccccctggatcaccgggatgctatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 156 aa, Artificial sequence
  Variant ligand binding domain of TREM2
  VARIANT (118)..(119): X at position 118 is not R and/or X at position 119 is not D
  VARIANT (139)..(140): X at position 139 is not H and/or X at position 140 is not S
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHXXAGDLWFPGESESFEDAHVEXXISRSLLEGEIPFPPTS

SEQ ID NO: 37 - protein, 156 aa, Artificial sequence
  Variant ligand binding domain of TREM2
  VARIANT (78)..(78): X is a basic amino acid, preferably K or R
  VARIANT (118)..(119): X at position 118 is not R and/or X at position 119 is not D
  VARIANT (139)..(140): X at position 139 is not H and/or X at position 140 is not S
HNTTVFQGVAGQSLQVSCPYDSMKHWGRRKAWCRQLGEKGPCQRVVSTHNLWLLSFLRRWNGSTAITDDTLGGTLTIXLRNLQPHDAGLYQCQSLHGSEADTLRKVLVEVLADPLDHXXAGDLWFPGESESFEDAHVEXXISRSLLEGEIPFPPTS

SEQ ID NO: 38 - protein, 52 aa, Homo sapiens
ERTMPRIPTLKNLEDLVTEYHGNFSAWSGVSKGLAESLQPDYSERLCLVSEI

SEQ ID NO: 39 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 40 - protein, 355 aa, Artificial sequence
  TREM2-CD28-CD3zeta CAR
MALPVTALLLPLALLLHAARPHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAIEVLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 378 aa, Artificial sequence
  TREM2-CD8-CD28-CD3zeta CAR
MALPVTALLLPLALLLHAARPHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 355 aa, Artificial sequence
  TREM2mut-CD28-CD3zeta CAR
MALPVTALLLPLALLLHAARPHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAIEVLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 378 aa, Artificial sequence
  TREM2mut-CD8-CD28-CD3zeta CAR
MALPVTALLLPLALLLHAARPHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 460 aa, Artificial sequence
  TREM2-CD3zeta-2A-DAP10 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 45 - protein, 501 aa, Artificial sequence
  TREM2-CD3zeta-2A-DAP10-CD28 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRSKRSRLLHSDYMNMTPRRPGPTRKHYQPYAPPRDFAAYRS

SEQ ID NO: 46 - protein, 408 aa, Artificial sequence
  TREM2-CD28-2A-DAP12 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRSKRSRLLHSDYMNMTPRRPGPTRKHYQPYAPPRDFAAYRSARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 47 - protein, 521 aa, Artificial sequence
  TREM2-CD28-2A-DAP12-CD3zeta CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRSKRSRLLHSDYMNMTPRRPGPTRKHYQPYAPPRDFAAYRSARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 48 - protein, 487 aa, Artificial sequence
  TREM2-CD3zeta-2A-DAP12trunc-CD28 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRSKRSRLLHSDYMNMTPRRPGPTRKHYQPYAPPRDFAAYRS

SEQ ID NO: 49 - protein, 333 aa, Artificial sequence
  TREM2-2A-DAP12trunc CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAAT

SEQ ID NO: 50 - protein, 460 aa, Artificial sequence
  TREM2mut-CD3zeta-2A-DAP10 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 51 - protein, 408 aa, Artificial sequence
  TREM2mut-CD28-2A-DAP12 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRSKRSRLLHSDYMNMTPRRPGPTRKHYQPYAPPRDFAAYRSARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 52 - protein, 408 aa, Artificial sequence
  TREM2-2A-DAP12-CD28 CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYKRSKRSRLLHSDYMNMTPRRPGPTRKHYQPYAPPRDFAAYRS

SEQ ID NO: 53 - protein, 460 aa, Artificial sequence
  TREM2-2A-DAP10-CD3zeta CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 487 aa, Artificial sequence
  TREM2-2A-DAP12trunc-CD28-CD3zeta CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 55 - protein, 487 aa, Artificial sequence
  TREM2mut-2A-DAP12tr-CD28-CD3zeta CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 56 - protein, 425 aa, Artificial sequence
  TREM2-2A-DAP12tr-CD28-CD3tr CAR
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTIKLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRSKRSRLLHSDYMNMTPRRPGPTRKHYQPYAPPRDFAAYRSGLYNELQKDKMAEAYSEIGMKGERRRGKGHDGLYQGLSTATKDTYDALHMQ

SEQ ID NO: 57 - protein, 460 aa, Artificial Sequence; identical to SEQ ID NO: 44
  TREM2-CD3z-2A-DAP10_2A-GFP

SEQ ID NO: 58 - protein, 501 aa, Artificial Sequence; identical to SEQ ID NO: 45
  TREM2-CD3z-2A-DAP10-CD28_2A_GFP

SEQ ID NO: 59 - protein, 408 aa, Artificial Sequence; identical to SEQ ID NO: 46
  TREM2-CD28-2A-DAP12_2A_GFP

SEQ ID NO: 60 - protein, 477 aa, Artificial Sequence
  TREM2-CD3z-2A-DAP12trunc-CD28_2A_GFP
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDTRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRSKRSRLLHSDYMNMTPRRPGPTRKHYQPYAPPRDFAAYRS

SEQ ID NO: 61 - protein, 333 aa, Artificial Sequence; identical to SEQ ID NO: 49
  TREM2-2A-DAP12trunc_2A_GFP

SEQ ID NO: 62 - DNA, 1380 bp, Artificial Sequence
  TREM2-CD3z-2A-DAP10_2A-GFP
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcccgcgccaaaaggtctggctccggtgagggcagaggaagtcttataacatgcggtgacgtggaggagaatcccggccctatgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggc

SEQ ID NO: 63 - DNA, 1503 bp, Artificial Sequence
  TREM2-CD3z-2A-DAP10-CD28_2A_GFP
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcccgcgccaaaaggtctggctccggtgagggcagaggaagtcttataacatgcggtgacgtggaggagaatcccggccctatgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggcaggagtaagaggagcaggctcctgcacagtgactacatgaacatgactccccgccgccccgggcccacccgcaagcattaccagccctatgccccaccacgcgacttcgcagcctatcgctcc

SEQ ID NO: 64 - DNA, 1224 bp, Artificial Sequence
  TREM2-CD28-2A-DAP12_2A_GFP
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgaggagtaagaggagcaggctcctgcacagtgactacatgaacatgactccccgccgccccgggcccacccgcaagcattaccagccctatgccccaccacgcgacttcgcagcctatcgctccgcccgcgccaaaaggtctggctccggtgagggcagaggaagtcttataacatgcggtgacgtggaggagaatcccggccctatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 65 - DNA, 1431 bp, Artificial Sequence
  TREM2-CD3z-2A-DAP12trunc-CD28_2A_GFP
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcccgcgccaaaaggtctggctccggtgagggcagaggaagtcttataacatgcggtgacgtggaggagaatcccggccctatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctaggagtaagaggagcaggctcctgcacagtgactacatgaacatgactccccgccgccccgggcccacccgcaagcattaccagccctatgccccaccacgcgacttcgcagcctatcgctcc

SEQ ID NO: 66 - DNA, 999 bp, Artificial Sequence
  TREM2-2A-DAP12trunc_2A_GFP
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacggcccgcgccaaaaggtctggctccggtgagggcagaggaagtcttataacatgcggtgacgtggaggagaatcccggccctatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacc

SEQ ID NO: 67 - protein, 153 aa, Artificial sequence
  Mutant TREM2 ligand binding domain and stalk domain
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTS

SEQ ID NO: 68 - protein, 227 aa, Artificial sequence
  Mutant TREM2 exodomain
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHSISRSLLEGEIPFPPTSILLLLACIFLIKILAASALWAAAWHGQKPGTHPPSELDCGHDPGYQLQTLPGLRDT

SEQ ID NO: 69 - protein, 370 aa, Artificial sequence
  WT-TREM2-2A-DAP12-2A-GFP
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDTARAKRSGSGEGRGSLITCGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 70 - DNA, 1110 bp, Artificial sequence
  WT-TREM2-2A-DAP12-2A-GFP
atggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacggcccgcgccaaaaggtctggctccggtgagggcagaggaagtcttataacatgcggtgacgtggaggagaatcccggccctatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

(19) United States
(12) Patent Application Publication
(10) Pub. No.: US 2023/0235009 A1
(43) Pub. Date: Jul. 27, 2023

(54) TREM2 CHIMERIC RECEPTOR

(71) Applicants: QUELL THERAPEUTICS LIMITED, London (GB); KING'S COLLEGE LONDON, London (GB)

(72) Inventors: Marc MARTINEZ-LLORDELLA, London (GB); Alberto SANCHEZ-FUEYO, London (GB); Simon BORNSCHEIN, Cremlingen (DE)

(73) Assignees: QUELL THERAPEUTICS LIMITED, London (GB); KING'S COLLEGE LONDON, London (GB)

(21) Appl. No.: 18/001,277

(22) PCT Filed: Jun. 11, 2021
(86) PCT No.: PCT/GB2021/051464

§ 371 Date: Dec. 9, 2022

(30) Foreign Application Priority Data
Jun. 11, 2020 (GB) 2008855.5
Oct. 30, 2020 (GB) 2017263.1

(51) Int. Cl. C07K 14/725; C12N 15/63

(52) U.S. Cl. CPC C07K 14/7051; C12N 15/63; C07K 2319/03; C07K 2319/02

(57) ABSTRACT

The present invention relates to chimeric receptors (e.g. CARs including both single chain and multichain CARs) that bind to TREM2 ligands and their use in therapy. In particular, the invention provides a chimeric receptor comprising:
(a) an exodomain comprising the ligand binding domain of TREM2 or a functional variant thereof, optionally wherein said exodomain is resistant to cleavage by a sheddase;
(b) a transmembrane domain; and
(c) an endodomain comprising an intracellular signalling domain.

### FIELD OF THE INVENTION

The present invention relates generally to the field of chimeric receptors, TREM2 biology and related therapies, such as the treatment of neurological disorders characterized by neuronal damage, neuroinflammation or neurodegeneration. More particularly, the invention provides chimeric antigen receptors (CARs) (both single chain and multichain CARs) that bind to TREM2 ligands (e.g. ApoE, AR oligomers, etc) and that are expressed in immune cells (e.g. Tregs). Such immune cells have therapeutic uses in diseases and conditions associated with the accumulation of TREM2-expressing cells and/or soluble TREM2, or where TREM2 ligands are present and/or expressed. The invention further provides nucleic acid molecules encoding such CARs and vectors containing them that may be used to modify host cells, e.g. immune cells, to express the CARs.

### BACKGROUND TO THE INVENTION

Inflammation is the body's biological response to injury and infection and functions to eliminate the initial cause of cell injury and effect repair. However, an immune response that results in chronic inflammation can lead to tissue damage and ultimately its destruction. Chronic inflammation is often a result of an inappropriate immune response.

Inflammation in the nervous system (“neuroinflammation”) can be particularly harmful, especially when sustained for a long period. Although inflammation may not be disease-causing in and of itself, it can contribute to disease pathogenesis across both the peripheral nervous system (e.g. neuropathic pain, fibromyalgia) and the central nervous system (e.g., amyotrophic lateral sclerosis (ALS), Alzheimer disease, Parkinson disease, multiple sclerosis and other demyelinating diseases, ischemic and traumatic brain injury, depression, and autism spectrum disorder). Communication between the nervous system and immune system may represent an important factor in neuroinflammation.

Microglia are macrophage-like myeloid cells that play a critical role in maintaining the homeostasis of the nervous system by regulating cell death and neurogenesis and by contributing to synaptic pruning during postnatal development. A key property of microglia is their capacity to modify their activation status based on environmental changes. Thus, upon neuronal injury or in neurodegenerative/neuroinflammatory diseases, microglia become activated and exert protective effects such as phagocytosis of cell debris and secretion of neurotrophic mediators. However, under some circumstances, microglia amplify the damage of the nervous tissue by producing toxic molecules, releasing cytokines/chemokines, presenting antigens to T cells and phagocytosing the injured neurones. Considering that the proportion of the global population suffering from neuroinflammatory and neurodegenerative disorders is increasing, there is an urgent need for new therapies to modulate microglial activity and plasticity as a means to treat these diseases.

TREM2 is a type 1 transmembrane protein member of the Ig superfamily that is expressed on microglia (and other myeloid cell subsets), and binds anionic lipids and DNA released during neuronal and glial damage, as well as other molecules such as amyloid beta oligomers (Aβ). TREM2 forms a complex with DAP12, which, upon ligand binding to TREM2, transduces signals into the cytoplasm through its ITAM motifs. Whilst TREM2 has been reported to support microglial metabolism and to promote the migration, cytokine release, phagocytosis, proliferation and survival of the cells, the role of TREM2 within specific disease conditions is complicated. Particularly, whilst TREM2/DAP12 mediated microglial activation is detrimental for some diseases, it has been reported to be beneficial for other conditions, and the role of TREM2 as a pro or anti-inflammatory molecule is unclear. Thus, therapies based on targeting TREM2/DAP12 per se, which have been suggested by some groups, may only represent a specific approach to treat particular conditions. Alternative therapies to modulate microglia activation status and protect the damaged nervous tissue are therefore required in order to tackle a wide range of neuroinflammatory and neurodegenerative conditions.

CD4+Foxp3+ regulatory T cells (Tregs) are a lymphocyte subset that is essential for the maintenance of dominant immunological tolerance by inhibiting the function of various effector immune cell subsets, including myeloid cells such as macrophages and dendritic cells. In addition, Tregs are also known to promote tissue repair and regeneration. Tregs are known to be involved in controlling neuroinflammatory disorders such as multiple sclerosis, amyotrophic lateral sclerosis, as well as ischemic and traumatic brain injury. The prospect of ameliorating immunopathology and re-establishing tolerance in inflammatory diseases has prompted a growing interest in the clinical development of Treg-based immunotherapies. For a Treg immunotherapy to be successful it is essential to develop strategies that promote the trafficking of Tregs to the site of tissue damage and induce their activation in situ.

### SUMMARY OF THE INVENTION

The present inventors have determined that a generic therapy for the treatment of neurodegenerative and other conditions associated with the TREM2 pathway (i.e. associated with the accumulation of soluble TREM2 or TREM2-expressing cells) may be developed by providing immune cell subsets with a chimeric antigen receptor comprising the extracellular domain of TREM2 itself. Contrary to other direct TREM2 targeting approaches that have been suggested in the art, the inventors' approach of utilising the extracellular domain of TREM2 obviates the problem of unclarity regarding the role of TREM2 within particular conditions by not directly targeting the molecule or the cells upon which it is expressed. Particularly, the inventors have discovered that a functional CAR may be produced by using a modified extracellular domain of TREM2, to prevent cleavage of the domain by sheddase enzymes. More particularly, the expression of such a CAR on the surface of Tregs is likely to provide a generic therapy that can be used for the treatment of conditions associated with inflammation, where TREM2 is expressed locally at the site of disease, in view of the well-known bystander effect of Tregs and their ability, once activated, to reduce the immune response and modulate the activation status of myeloid cells and other immune cell subsets.

Accordingly, in one aspect, the present invention provides a chimeric receptor comprising:

(a) an exodomain comprising the ligand binding domain of TREM2 or a functional variant thereof;

(b) a transmembrane domain; and

(c) an endodomain comprising an intracellular signalling domain.

In a particular aspect, the present invention provides a chimeric receptor comprising:

(a) an exodomain comprising the ligand binding domain of TREM2 or a functional variant thereof, wherein said exodomain is resistant to cleavage by a sheddase;

(b) a transmembrane domain; and

(c) an endodomain comprising an intracellular signalling domain.ln a further aspect, the invention provides a nucleic acid molecule encoding the chimeric receptor of the invention.

In another aspect, the invention provides a vector comprising the nucleic acid molecule of the invention.

The present invention also provides a cell (e.g. an immune cell) which expresses the chimeric receptor of the invention and/or a cell (e.g. an immune cell) which comprises a nucleic add molecule encoding a chimeric receptor of the invention. The cell may be provided in a cell population, which forms a further aspect of the invention.

In another aspect the invention provides a pharmaceutical composition comprising a cell or cell population of the invention.

The present invention further provides a method for producing a cell of the invention comprising introducing a nucleic acid or vector of the invention into a cell.

The present invention further provides a method for treating and/or preventing a disease or condition (e.g. a neurological disease or condition) in a subject, which comprises the step of administering a cell, cell population or pharmaceutical composition according to the invention to the subject.

Alternatively viewed, the invention provides a cell, cell population or pharmaceutical composition according to the invention for use in therapy (e.g. treating and/or preventing a neurological disease or condition).

### DETAILED DESCRIPTION

The term “chimeric receptor” refers to a receptor protein comprising linked domains from two or more proteins, e.g. an exodomain from a first protein and an endodomain from a second protein. Typically, at least one of the domains is derived from a receptor protein. Thus, a chimeric receptor may be viewed as an “engineered receptor” and these terms are used interchangeably herein.

A chimeric receptor may comprise linked domains on a single polypeptide chain (a single contiguous chain) or may comprise two or more polypeptide chains (a multichain chimeric receptor), wherein at least one of the polypeptide chains comprises linked domains from two or more proteins. Hence, a chimeric receptor of the invention which is a multichain receptor may comprise a first polypeptide chain and a second polypeptide chain. Thus, a chimeric receptor may comprise at least two polypeptide chains which may associate with each other when co-expressed, particularly through their transmembrane domains, and/or through an alternative dimerization site. Typically, each polypeptide chain within a multichain chimeric receptor will comprise of two or more linked domains, for example, a first polypeptide chain may comprise an extracellular domain and a transmembrane domain, and a second polypeptide may comprise a transmembrane domain and an endodomain, or a first polypeptide may comprise an extracellular domain, a transmembrane domain and an endodomain and a second polypeptide may comprise a transmembrane domain and an endodomain. However, it is also possible for one of the polypeptide chains to only comprise a single domain, typically an endodomain. It will therefore be appreciated that a chimeric receptor of the invention may comprise more than one of a particular domain within the same or within different polypeptide chains. For example, where the chimeric receptor is a multichain chimeric receptor, the chimeric receptor may comprise two transmembrane domains and/or two endodomains which may be the same or different.

A “Chimeric antigen receptor”, “CAR” or “CAR construct” refers to engineered receptors which can confer an antigen specificity onto cells (e.g. immune cells, such as Tregs). As discussed above, a CAR may comprise a single polypeptide chain or may comprise two or more polypeptide chains (e.g. a first polypeptide chain and a second polypeptide chain). In particular, a CAR enables a cell to bind specifically to a particular antigen, e.g. a target molecule such as a target protein, whereupon a signal is generated by the endodomain (comprising an intracellular signalling domain) of the CAR, e.g. a signal resulting in activation of the cell. CARs are also known as artificial T-cell receptors, chimeric T-cell receptors or chimeric immunoreceptors. Thus, as the chimeric receptor of the invention functions to confer cells expressing the receptor (e.g. Tregs) with the ability to bind specifically to TREM2 ligands, such as ApoE, it may be viewed as a CAR.

The structure of CARs is well-known in the art and several generations of CARs have been produced. For instance, as a minimum a CAR may contain an extracellular antigen-specific targeting region, antigen binding domain or ligand binding domain, which is or forms part of the exodomain (also known as the extracellular domain or ectodomain) of the CAR, a transmembrane domain, and an intracellular signalling domain (which is or is comprised within an endodomain). However, the CAR may contain further domains to improve its functionality, e.g. one or more co-stimulatory domains to improve T cell proliferation, cytokine secretion, resistance to apoptosis, and in vivo persistence. As discussed above, the CAR may comprise more than one polypeptide chain and thus the domains may occur within the same or within different polypeptides, typically which associate with one another. Thus, in one embodiment, a CAR may comprise two polypeptides wherein the first polypeptide comprises the extracellular domain, a transmembrane domain and optionally an endodomain, and the second polypeptide comprises an endodomain and optionally a transmembrane domain. Particularly, at least one endodomain in a multichain CAR will comprise an intracellular signalling domain.

Thus, a CAR construct generally comprises an antigen or ligand binding domain, optionally a hinge domain, which functions as a spacer to extend the antigen or ligand binding domain away from the plasma membrane of the cell (e.g. immune cell) on which it is expressed, a transmembrane domain, an intracellular signalling domain (e.g. the signalling domain from the zeta chain of the CD3 molecule (CD3) of the TcR complex, or an equivalent) and optionally one or more co-stimulatory domains, which may assist in signalling or functionality of the cell expressing the CAR. A CAR may also comprise a signal or leader sequence or domain which functions to target the protein to the membrane and may form part of the exodomain of the CAR. The different domains may be linked directly or by linkers, and/or may occur within different polypeptides, e.g. within two polypeptides which associate with one another. A variety of options are available for these different domains and linkers as discussed in detail below.

The exodomain of the chimeric receptor of the invention contains an antigen or ligand binding domain comprising the ligand binding domain of TREM2 or a functional variant thereof. The exodomain may be resistant to cleavage by a sheddase.

The human isoform 1 of TREM2 (SEQ ID NO: 1) comprises an exodomain (amino acids 1-174 of SEQ ID NO: 1), a transmembrane domain (amino acids 175-195 of SEQ ID NO: 1) and an endodomain (amino acids 196-230 of SEQ ID NO: 1). The exodomain of TREM2 (SEQ ID NO: 2) comprises at least three domains: a signal or leader sequence (amino acids 1-18 of SEQ ID NO: 2); a ligand binding domain (amino acids 19-130 of SEQ ID NO: 2); and a stalk region (amino acids 131-174 of SEQ ID NO: 2). The ligand binding domain and the stalk region have a sequence as set out in SEQ ID NO. 3 (amino acids 19-174 of SEQ ID NO. 2). The ligand binding domain of the exodomain comprises three complementary determining regions (CDR1: amino acids 38-47 of SEQ ID NO: 1; CDR2: amino acids 65-75 of SEQ ID NO: 1; and CDR3: amino acids 88-91 of SEQ ID NO: 1). The exodomain of TREM2 also comprises a dipeptide sheddase cleavage site (amino acids 157-158 of SEQ ID NO: 1), which is cleaved by a Disintegrin and metalloproteinase domain-containing protein (ADAM) 10 and, to a lesser extent, ADAM17. The exodomain of TREM2 may further comprise a meprin beta cleavage site between amino acids 136-137 of SEQ ID NO. 1.

Thus, as discussed above, the exodomain of the chimeric receptor of the invention comprises the ligand binding domain of the TREM2 exodomain (which may comprise amino acid residues 19-130 of SEQ ID NO. 2), or a functional variant thereof. The exodomain may be resistant to cleavage by a sheddase. Thus, in one embodiment, the exodomain may consist of the ligand binding domain of the TREM2 exodomain (amino acid residues 19-130 of SEQ ID NO. 2) or a functional variant thereof or may comprise additional sequence. It will be appreciated that in accordance with the invention, when an additional sequence is present (e.g. between the ligand binding domain of the TREM2 exodomain (amino acids 19-130 of SEQ ID NO. 2) or a functional variant thereof and the transmembrane domain of the chimeric receptor of the invention), the additional sequence may correspond to the wild-type sequence of TREM2 or to a functional variant thereof and may be resistant to cleavage by a sheddase. The exodomain may comprise any additional sequence (e.g. between the ligand binding domain of the TREM2 exodomain (amino acid residues 19-130 of SEQ ID NO. 2) or a functional variant thereof and the transmembrane domain), where the sequence may be resistant to cleavage by a sheddase. Particularly, the exodomain may comprise the stalk region of the exodomain of TREM2 (amino acids 131-174 of SEQ ID NO. 2) or a variant of the stalk region, wherein said variant of the stalk region may be resistant (e.g. fully or partially resistant) to cleavage by a sheddase (e.g. by truncation and/or by mutation), or may not be resistant to cleavage by a sheddase. For example, the exodomain may comprise amino acids 131-156 or 157 of SEQ ID NO. 2, or amino acids 131-135 or 136 of SEQ ID NO. 2. In such embodiments, the exodomain may comprise amino acid residues 19-156 or 157 of SEQ ID NO. 2 or amino acid residues 19-135 or 136 of SEQ ID NO. 2, or a functional variant thereof. It will be appreciated that the use of truncated variants of the TREM2 stalk will remove cleavage sites which naturally occur within TREM2 to prevent their occurrence in the exodomain of a CAR of the invention. Alternatively, or additionally, any naturally occurring cleavage sites may be removed by mutation, such as by deletion mutation, or by protection of the cleavage site.

Accordingly, in some embodiments the exodomain of the chimeric receptor of the invention comprises:

(i) an amino acid sequence as set forth in SEQ ID NO: 3; or

(ii) a functional variant of the amino acid sequence as set forth in SEQ ID NO: 3. The functional variant of the exodomain may be resistant to cleavage by a sheddase.

The term “functional variant” refers to variants of the TREM2 exodomain (e.g. of SEQ ID NO. 2 or SEQ ID NO. 3), and particularly to variants of the ligand binding domain thereof (e.g. comprising amino acid residues 19-130 of SEQ ID NO. 2), that are capable of binding specifically to a TREM2 ligand, particularly ApoE, cell debris, dead or dying (i.e. apoptotic/necrotic) cells, e.g. with the same, similar or greater affinity as TREM2. Thus, the functional variant of the TREM2 exodomain or of the ligand binding domain of the TREM2 exodomain of the chimeric receptor functions to enable the chimeric receptor to bind specifically to a TREM2 ligand, particularly ApoE, cell debris or dead or dying (i.e. apoptotic/necrotic) cells.

By “similar affinity” is meant that the binding affinity of the functional variant for a TREM2 ligand (e.g. human TREM2 ligand) is comparable to the TREM2 receptor, e.g. is not more than a factor of 20 different. More preferably the difference between the binding affinities is less than a factor of 15, more preferably less than a factor of 10, most preferably less than a factor of 5, 4, 3 or 2.

In particular, the functional variant of the TREM2 exodomain and particularly of the ligand binding domain of the TREM2 exodomain (e.g. residues 19-130 of SEQ ID NO. 2), which forms the antigen/ligand binding domain of the chimeric receptor (which itself forms part of the exodomain of the chimeric receptor) is capable of binding specifically to a TREM2 ligand (e.g. ApoE or cell debris), particularly when the chimeric receptor is expressed on the surface of a cell (e.g. an immune cell). Specific binding may be distinguished from non-specific binding to a non-target antigen (in this case an antigen other than a TREM2 ligand, such as an antigen other than ApoE). Thus, a cell, particularly an immune cell (e.g. Treg), expressing the chimeric receptor according to the present invention is directed to bind specifically to a TREM2 ligand, particularly to ApoE.

In some embodiments, specific binding to a TREM2 ligand (e.g. ApoE) may mean that the functional variant of the TREM2 exodomain or of the TREM2 ligand binding domain, e.g. having residues 19-130 of SEQ ID NO. 2, or the chimeric receptor comprising the functional variant of the TREM2 exodomain or of the TREM2 ligand binding domain, binds to, or associates with, a TREM2 ligand, with an affinity or Ka (i.e. equilibrium association constant) of greater than or equal to about 10⁵ M⁻¹, e.g. at least about 10⁶ M⁻¹, 10⁷ M⁻¹, or 10⁸ M⁻¹.

Variants of the TREM2 ligand binding domain with increased affinity for a TREM2 ligand, e.g. ApoE, are known in the art and any such variant may be used in the chimeric receptor of the invention. Thus, in some embodiments, the exodomain of the chimeric receptor comprises a functional variant of the ligand binding domain of the TREM2 exodomain with increased affinity for a TREM2 ligand, preferably ApoE.

In particular, it has been shown that mutation of the tyrosine residue at position 96 of SEQ ID NO: 1 to lysine, increases the binding affinity of the exodomain for ApoE. Thus, in some embodiments, the functional variant of the ligand binding domain of the TREM2 exodomain comprises substitution of the tyrosine at position 96 with a basic amino acid residue, e.g. lysine or arginine, preferably lysine.

Thus, in some embodiments, the exodomain of the chimeric receptor of the invention comprises:

an amino acid sequence as set forth in SEQ ID NO: 4 or a functional variant thereof

wherein the amino acid at the position equivalent to position 78 of SEQ ID NO: 4 (i.e. equivalent to position 96 of SEQ ID NOs: 1 and 2) is a basic amino acid, preferably lysine or arginine, and wherein the exodomain may be resistant to cleavage by a sheddase. Particularly, the ligand binding domain of the chimeric receptor of the invention consists of an amino acid sequence as set forth in SEQ ID NO. 4.

It will be appreciated that a functional variant as referred to herein may comprise at least one alteration (e.g. at least one amino acid substitution, deletion, and/or addition etc) within the ligand binding domain of the TREM2 exodomain (e.g. within amino acids 19-130 of SEQ ID NO. 2), and/or at any other position within the TREM2 exodomain where additional TREM2 sequence is present, as long as the variant retains the functions described above, i.e. specific binding to a TREM2 ligand and optionally resistance to a cleavage by a sheddase. Thus, particularly, where the exodomain of the chimeric receptor comprises a functional variant of SEQ ID No 3, the variant may comprise at least one amino acid substitution, deletion or addition within the ligand binding domain of the exodomain of TREM2 (i.e. within amino acids 19-130 of SEQ ID NO. 2, e.g. as shown in SEQ ID NO. 4) and/or at least one amino acid substitution, deletion and/or addition within the stalk of the exodomain of TREM2 (within amino acid residues 131-174 of SEQ ID NO. 2). As discussed in further detail below, particularly such a functional variant of SEQ ID No 3 may comprise a deletion (e.g. a truncation) or substitution of any amino acid residues which are capable of being cleaved by a sheddase (particularly within the stalk region e.g. within amino acid residues 131-174 of SEQ ID NO. 2).

As discussed above, the ligand binding domain of TREM2 comprises three CDRs; CDR1 (amino acid residues 38-47 of SEQ ID NO. 1), CDR2 (amino acid residues 65-75 of SEQ ID NO. 1) and CDR3 (amino acid residues 88-91 of SEQ ID NO. 1). In a particular embodiment of the invention, a functional variant of the ligand binding domain of TREM2 may comprise the wildtype sequences of CDRs1-3 or these regions may have only minor modifications, e.g. one, two, or three amino acid substitutions, particularly conservative amino acid substitutions. Thus, a functional variant of the ligand binding domain of TREM2 may particularly comprise modifications outside of the CDR regions, e.g. within amino acids 19-37, 48-64, 76-87 and/or 92-130 of SEQ ID NO. 2.

In this regard, the invention also provides a chimeric receptor comprising:

(a) an exodomain comprising a ligand binding domain comprising CDR 1 (amino acid residues 38-47 of SEQ ID NO. 1), CDR2 (amino acid residues 65-75 of SEQ ID NO. 1) and CDR3 (amino acid resides 88-91 of SEQ ID NO. 1) of TREM2 or a functional variant thereof having one, two or three amino acid substitutions (particularly conservative amino acid substitutions) within CDR1, 2 and/or 3, wherein said exodomain may be resistant to cleavage by a sheddase;

(b) a transmembrane domain; and

(c) an endodomain comprising an intracellular signalling domain.

The term “resistant to cleavage by a sheddase” means that the exodomain of the chimeric receptor of the invention is not substantially cleaved (e.g. not fully cleaved or only partially cleaved) when contacted with a sheddase enzyme under suitable conditions, i.e. conditions suitable for the sheddase enzyme to function. In some embodiments, less than about 80%, e.g. less than about 80%, 75%, 70%, 65%, 60%, 55%, 50%, 45%, 40%, 35%, 30%, 25%, 20%, 15% or 10% of the exodomain of the chimeric receptor is cleaved by the sheddase enzyme, preferably less than about 60%, e.g. less than 40%, less than 30%, less than 20%, less than 10%, more preferably less than about 5%, e.g. less than 4, 3, 2, 1, 0.5 or 0.1% is cleaved.

Cleavage of the exodomain of the chimeric receptor of the invention may be measured using any suitable means known in the art. For instance, a cell expressing the chimeric receptor of the invention may be contacted with a sheddase enzyme under conditions suitable for activity of the sheddase for a specified time and the amount of exodomain cleaved from the chimeric receptor may be measured, e.g. by SDS-PAGE, ELISA etc. Conveniently, the amount of exodomain cleaved from the chimeric receptor of the invention may be compared to the amount of exodomain cleaved from a cell expressing an equivalent chimeric receptor comprising an unmodified (wild-type) TREM2 exodomain contacted with a sheddase enzyme under the same conditions and for the same amount of time. The exodomain of the chimeric receptor of the invention may be viewed as resistant to cleavage by a sheddase enzyme when less than about 80%, e.g. less than about 80%, 75%, 70%, 65%, 60%, 55%, 50%, 45%, 40%, 35%, 30%, 25%, 20%, 15% or 10% of the exodomain of the chimeric receptor is cleaved by the sheddase enzyme, preferably less than about 60%, e.g. less than 40%, less than 30%, less than 20%, less than 10%, more preferably less than about 5%, e.g. less than 4, 3, 2, 1, 0.5 or 0.1% is cleaved.

Contacting a cell expressing the chimeric receptor with a sheddase enzyme may be achieved by expressing a sheddase enzyme in the same cell as the chimeric receptor. In some embodiments, expression of the sheddase enzyme may be under the control of an inducible promoter such that contacting the cell with a sheddase may involve incubating the cell with an agent to induce expression of the sheddase enzyme for a defined period of time. In some embodiments, contacting a cell expressing a chimeric receptor with a sheddase enzyme may be achieved by contacting the cell expressing a chimeric receptor with a cell expressing a sheddase enzyme.

The terms “sheddase” and “sheddase enzyme” are used interchangeably herein and refer to membrane-bound enzymes that cleave extracellular domains of transmembrane proteins. In some embodiments, the sheddase is a member of the ADAM (a disintegrin and metalloproteinase) protein family such as a protein in enzyme group EC 3.4.24.81 (e.g. ADAM 10) or ADAM17. In other embodiments, the sheddase is a member of the metalloproteinases, such as meprin β.

Thus, in some embodiments, the exodomain of the chimeric receptor of the invention is resistant to cleavage by one or more members of the ADAM protein family, e.g. a protein in enzyme group EC 3.4.24.81, and/or by one or more members of the metalloproteinases, e.g. meprin 13. In a preferred embodiment, the exodomain of the chimeric receptor of the invention is resistant to cleavage by ADAM10 and/or ADAM 17, preferably ADAM10. In another embodiment, the exodomain of the chimeric receptor of the invention is resistant to cleavage by meprin β.

Suitable conditions for a sheddase enzyme may be any conditions under which the enzyme is able to cleavage its target protein efficiently. The skilled person would readily be able to determine suitable conditions for sheddase activity.

The inventors have advantageously determined that a chimeric receptor capable of binding specifically to a TREM2 ligand (e.g. ApoE, cell debris or apoptotic or necrotic cells) may be produced using wild-type TREM2 or a variant (e.g. truncated and/or mutated) TREM2 exodomain, i.e. a functional variant (e.g. portion) of the TREM2 exodomain, e.g. a functional variant that is resistant to cleavage by a sheddase. In particular, the inventors have identified a portion of the TREM2 exodomain that is sufficient to produce a chimeric receptor capable of binding to a TREM2 ligand (e.g. ApoE, cell debris or apoptotic or necrotic cells), whilst being resistant to cleavage by a sheddase enzyme.

In a preferred embodiment, the exodomain of the chimeric receptor comprises the ligand binding domain of the TREM2 exodomain, e.g. amino acids 19-130 of SEQ ID NO: 2, or a functional variant thereof. It will be appreciated that such a portion of the TREM2 exodomain may not contain the dipeptide sheddase cleavage site(s). In some embodiments, the exodomain of the chimeric receptor additionally comprises a portion of the stalk domain of TREM2 and/or a variant of the stalk domain of TREM2, that may not contain a dipeptide sheddase cleavage site (e.g. at 157-158, (e.g. amino acids 19-156 of SEQ ID NO: 2), or at 136-137, (e.g. amino acids 19-135 of SEQ ID NO.2)). In some embodiments, the exodomain of the chimeric receptor comprises amino acids 19-138 of SEQ ID NO: 2, or a functional variant thereof (e.g. wherein the cleavage site 136-137 of SEQ ID NO. 2 has been modified or deleted). In other embodiments, the exodomain of the chimeric receptor comprises amino acids 19-135 of SEQ ID NO. 2, or a functional variant thereof.

Thus, in some embodiments, the exodomain of the chimeric receptor of the invention comprises or consists of:

an amino acid sequence as set forth in SEQ ID NO: 5 or 6 or a functional variant thereof

wherein the amino acid at the position equivalent to position 78 of SEQ ID NO: 6 (i.e. equivalent to position 96 of SEQ ID NOs: 1 and 2) is a basic amino acid, preferably lysine or arginine, and wherein said exodomain may be resistant to cleavage by a sheddase.

In some embodiments, the exodomain of the chimeric receptor of the invention comprises a functional variant of the TREM2 exodomain that has been modified, e.g. mutated, to make the exodomain resistant to cleavage by a sheddase enzyme. For instance, one or both of the residues that form the dipeptide sheddase cleavage domain (amino acids at positions 157-158 of SEQ ID NO: 1 and 2, and/or amino acids at positions 136-137 of SEQ ID Nos 1 and 2) may be substituted, deleted or a combination thereof to make the exodomain resistant to cleavage by a sheddase enzyme. In one embodiment, the functional variant of the TREM2 exodomain may comprise a C-terminal truncation to remove amino acids at positions 157-158 and a mutation to one or more of the amino acids at positions 136-137 of SEQ ID Nos 1 or 2 to provide an exodomain resistant to cleavage by a sheddase.

Thus, in some embodiments, the exodomain of the chimeric receptor of the invention comprises or consists of:

an amino acid sequence as set forth in SEQ ID NO: 7 or 8 or a functional variant thereof;

wherein

(a) the amino acid at the position equivalent to position 78 of SEQ ID NO: 8 (i.e. equivalent to position 96 of SEQ ID NOs: 1 and 2) is a basic amino acid, preferably lysine or arginine;

(b) the amino acids at positions equivalent to positions 139-140 of SEQ ID NO: 7 or 8 (i.e. equivalent to positions 157-158 of SEQ ID NO:2) are:
(1) not histidine and/or serine, respectively; and/or
(2) modified to make the exodomain resistant to cleavage by a sheddase, and optionally

(c) the amino acids at the position equivalent to positions 118-119 of SEQ ID NO 7 or 8 (i.e. equivalent to positions 136-137 of SEQ ID NO. 2) are:

(1) not arginine and/or aspartic acid, respectively; and/or

(2) modified to make the exodomain resistant to cleavage by a sheddase. Thus, the exodomain defined above may be resistant to cleavage by a sheddase.

In a preferred embodiment, the amino acid at the position equivalent to 139 of SEQ ID Nos 7 or 8 (i.e. equivalent to position 157 of SEQ ID NO. 2) is not tyrosine.

Thus, in some embodiments, the exodomain of the chimeric receptor of the invention comprises or consists of:

an amino acid sequence as set forth in SEQ ID NO: 36 or 37 or a functional variant thereof;

wherein

(a) the amino acid at the position equivalent to position 78 of SEQ ID NO: 37 (i.e. equivalent to position 96 of SEQ ID NOs: 1 and 2) is a basic amino acid, preferably lysine or arginine; and

(b) the amino acids at positions equivalent to positions 139-140 of SEQ ID NO: 36 or 37 (i.e. equivalent to positions 157-158 of SEQ ID NO:2) are:
(1) not histidine (and preferably is not tyrosine) and/or serine, respectively; and/or
(2) modified to make the exodomain resistant to cleavage by a sheddase; and

(c) the amino acids at the position equivalent to positions 118-119 of SEQ ID NO 36 or 37 (i.e. equivalent to positions 136-137 of SEQ ID NO. 2) are:

(1) not arginine and/or aspartic acid, respectively; and/or

(2) modified to make the exodomain resistant to cleavage by a sheddase.

Thus, the exodomain defined above may be resistant to cleavage by a sheddase.

In some embodiments, one or both of the amino acids at positions equivalent to positions 139-140 of SEQ ID NO: 7 or 8 (i.e. equivalent to positions 157-158 of SEQ ID NO:2) and/or at positions equivalent to positions 118-119 of SEQ ID Nos 7 or 8 (i.e. equivalent to positions 136-137 of SEQ ID NO. 2) are conservative or non-conservative substitutions relative to the amino acids in the wild-type sequence, preferably non-conservative substitutions.

In some embodiments, one or both of the amino acids at positions equivalent to positions 139-140 of SEQ ID NO: 7 or 8 (i.e. equivalent to positions 157-158 of SEQ ID NO:2) and/or at positions equivalent to positions 118-119 of SEQ ID Nos 7 or 8 (i.e. equivalent to positions 136-137 of SEQ ID NO. 2) are deleted.

Thus, in some embodiments, the exodomain of the chimeric receptor of the invention comprises or consists of an amino acid sequence as set forth in SEQ ID NO: 67 or 68 or a functional variant thereof, e.g. an amino acid sequence with at least 80% sequence identity to SEQ ID NO: 67 or 68.

In the functional variant of SEQ ID NO: 67 or 68 the amino acids at positions equivalent to positions 139-140 of SEQ ID NO: 67 or the amino acids at positions equivalent to positions 157-158 of SEQ ID NO: 68 (i.e. equivalent to positions 157-158 of SEQ ID NO:2) are serine and arginine, respectively or conservative or non-conservative substitutions thereof other than histidine and serine, respectively.

The functional variant of SEQ ID NO: 67 or 68 may contain the other modified residues specified above.

In some embodiments, amino acid residues outside of the dipeptide sheddase cleavage site(s) may be mutated (e.g. deleted or substituted) and these mutations may confer resistance to a sheddase enzyme. These mutations may be in addition, or an alternative, to the mutations described above. In some embodiments, one or more mutations (e.g. 2, 3, 4, 5, 6, 7, 8, 9 or 10 mutations) within 10 amino acids of a dipeptide sheddase cleavage site (i.e. in residues equivalent to 147-156 and/or 159-168 of SEQ ID NO: 2, and/or in residues equivalent to 125-135 and/or 138-148 of SEQ ID NO. 2) may confer resistance to a sheddase enzyme.

A conservative amino acid substitution refers to the replacement of an amino acid by another which preserves the physicochemical character of the polypeptide (e.g. H may be replaced by R or K or vice versa, S by T or vice versa). Thus, in some embodiments, the substituting amino acid has similar properties, e.g. hydrophobicity, hydrophilicity, electronegativity, bulky side chains etc. to the amino acid being replaced.

A non-conservative amino acid substitution refers to the replacement of an amino acid by another which does not preserve the physicochemical character of the polypeptide (e.g. H may be replaced by E or D, S by V, L, I, W etc). Thus, in some embodiments, the substituting amino acid has different properties, e.g. hydrophobicity, hydrophilicity, electronegativity, bulky side chains etc. to the amino acid being replaced.

In some embodiments, a functional variant of the present invention may differ from the recited sequences by, for example, 1 to 50, 1 to 45, 1 to 40, 1 to 35, 1 to 30, 1 to 25, 1 to 20, 1 to 15, 1 to 10, 1 to 8, 1 to 6, 1 to 5, 1 to 4, e.g. 1, 2 or 3 amino acid substitutions, insertions and/or deletions, preferably 1 to 23, 1 to 20, 1 to 15, 1 to 10, 1 to 8, 1 to 6, 1 to 5, 1 to 4, e.g. 1, 2 to 3 amino acid substitutions and/or 1 to 33, 1 to 30, 1 to 25, 1 to 20, 1 to 15, 1 to 10, 1 to 8, 1 to 6, 1 to 5, 1 to 4, e.g. 1, 2 or 3 amino acid deletions. As discussed below, in some embodiments, it is preferred that deletions are at the N- and/or C-terminus, i.e. truncations, thereby generating portions of the sequences defined above. As discussed previously, a skilled person will appreciate that whilst a functional variant may differ from the recited sequences as detailed above, preferably, fewer modifications may be made to the CDRs of the ligand binding domain (e.g. to CDRs 1, 2 and 3 of the TREM2 ligand binding domain). Modifications within the CDR regions may preferably be conservative amino acid substitutions or particularly, the CDR sequences may be retained unmodified. Amino acid modifications may particularly occur outside of the CDR regions, and most particularly to any TREM2 stalk which is included within the exodomain of the chimeric receptor.

The functional variants therefore encompass mutant forms of the TREM2 exodomain (i.e. referred to herein as homologues, variants or derivatives), which are structurally similar to the wild-type TREM2 exodomain set forth in SEQ ID NO: 2. The functional variants may be resistant to cleavage by a sheddase.

In cases where functional variants comprise mutations, e.g. deletions or insertions, relative to the recited sequences, the residues specified above are present at equivalent amino acid positions in the variant sequences. In some embodiments, deletions in the variants of the invention are not N-terminal and/or C-terminal truncations.

However, as mentioned above, it is contemplated that the functional variants described above may be truncated at the N-terminus and/or C-terminus without significantly reducing the binding affinity for a TREM2 ligand, e.g. ApoE, cell debris or apoptotic/necrotic cells. Thus, in some embodiments, the sequences recited above (e.g. of SEQ ID NO. 1 or 2) may be truncated by up to 20 amino acids at the N-terminus (e.g. 5, 10 or 15 amino acids) and/or by up to 36 amino acids at the C-terminus (e.g. 5, 10, 15, 20, 25, 30 or 35 amino acids). Thus, the term variant as used herein includes truncation variants of the exemplified polypeptides. Particularly preferred truncations (portions) are defined above.

In some embodiments, a “portion” comprises at least 90, 95, 100, 105, 110, 120 or more amino acids of sequences defined above (e.g. of any one of more of SEQ ID NOs. 1 to 8, 36 or 37). Thus, said portions may be obtained from a central or N-terminal or C-terminal portion of the sequence (e.g. of any one or more of SEQ ID Nos 1 to 8, 36 or 37). Preferably said portion is obtained from the central portion (e.g. of SEQ ID NO. 2), i.e. it comprises an N-terminal and/or C-terminal truncation as defined above. Notably, “portions” as described herein are polypeptides of the invention and therefore satisfy the identity (relative to a comparable region) conditions and functional equivalence conditions mentioned herein.

In some embodiments, a functional variant of the present invention may differ from the recited sequences by, for example, 1 to 5, 1 to 4, e.g. 1, 2 to 3 amino acid substitutions, insertions and/or deletions, preferably substitutions, as defined above. In some embodiments, the variant of the present invention may differ from SEQ ID NO: 2 as defined above.

Sequence identity may be determined by any suitable means known in the art, e.g. using the SWISS-PROT protein sequence databank using FASTA pep-cmp with a variable pamfactor, and gap creation penalty set at 12.0 and gap extension penalty set at 4.0, and a window of 2 amino acids. Other programs for determining amino acid sequence identity include the BestFit program of the Genetics Computer Group (GCG) Version 10 Software package from the University of Wisconsin. The program uses the local homology algorithm of Smith and Waterman with the default values: Gap creation penalty−8, Gap extension penalty=2, Average match=2.912, Average mismatch=−2.003.

In some embodiments, the functional variants and/or portions of the invention (e.g. amino acid residues 19-130, 19-135, 19-136, 19-137, 19-138, 19-156, 19-157, or 19-158 of SEQ ID NO. 2) have at least 70, 75, 80, 85, 90, 95, 96, 97, 98 or 99% sequence identity to a sequence as defined herein to which it is compared, e.g. a wild-type sequence of TREM2 or a portion thereof, e.g. one of SEQ ID NOs: 1-3 or 5.

Preferably said comparison is made over the full length of the sequence, but may be made over a smaller window of comparison, e.g. less than 100, 80 or 50 contiguous amino acids.

Preferably the variants (e.g. sequence identity-related variants) are functionally equivalent to the wild-type exodomain with respect to binding affinity for a TREM2 ligand, e.g. ApoE, cell debris, apoptotic or necrotic cells, as defined above.

In some embodiments, an equivalent position in the polypeptide of the invention is determined by reference to the amino acid sequence of SEQ ID NO: 1 or 2. The homologous or corresponding position can be readily deduced by lining up the sequence of the homologue (mutant, variant or derivative) polypeptide and the sequence of SEQ ID NO: 1 or 2 based on the homology or identity between the sequences, for example using a BLAST algorithm.

The terms “TREM2 ligand” or “TREM2-L” refer to any ligand to which TREM2 binds specifically via its extracellular domain, particularly via its ligand binding domain and thus any ligand to which the chimeric receptor of the invention may bind. TREM2 has been reported to bind to phosphatidylserine (PS), phosphatidylethanolamine (PE), phosphatidylcholine (PC), sulfoglycolipid, apolipoproteins, low-density lipoprotein, high-density lipoprotein, heat shock protein 60, DNA, E. coli, apoptotic and necrotic cells, cell debris, and Amyloid β peptide. Thus, the chimeric receptor of the invention, particularly the exodomain thereof, may bind to any one or more of the above ligands. In a particularly preferred embodiment, the exodomain of the chimeric receptor (and therefore the chimeric receptor) binds specifically to ApoE, i.e. any functional portion and/or variant of the exodomain must bind specifically to at least ApoE.

The binding of the ligand binding domain (e.g. exodomain) of the chimeric receptor to its target antigen (TREM2 ligand, e.g. ApoE) delivers an activation stimulus to the chimeric receptor-containing cell (e.g. Treg), resulting in induction of cell signalling pathways. Binding to the target antigen may thereby trigger proliferation, cytokine production, lytic activity and/or production of molecules that can mediate effects of the cell, e.g. the immunosuppressive effects provided by Tregs. Although chimeric receptors comprising an intracellular domain comprising solely a signalling domain from CD3 or FcRy may deliver a potent signal for immune cell activation and function they may not be sufficient to elicit signals that promote immune effector cell survival and expansion in the absence of a concomitant co-stimulatory signal. Accordingly, it may be preferred for the chimeric receptor to contain one or more co-stimulatory signalling domains.

A CAR of the invention thus generally comprises at least 3, 4, or 5, domains as follows:

(1) an exodomain comprising a ligand binding domain as defined above;

and optionally a hinge domain that extends the ligand binding domain away from the surface of the cell (e.g. the immune cell);

(2) a transmembrane domain that anchors the chimeric receptor to the cell and links the exodomain comprising the antigen binding domain to an endodomain (if present or if present on the same polypeptide chain);

(3) an endodomain comprising an intracellular signalling domain; and optionally or preferably;

one or more co-stimulatory signalling domains.

The domains of the CAR may occur within a single contiguous polypeptide, or may be present within two (or more) other polypeptide chains which associate with one another to provide signalling into the cell upon binding of the chimeric antigen receptor to its target antigen.

The chimeric receptor may further comprise a signal sequence (i.e. a targeting domain), and in particular, a sequence which targets the chimeric receptor to the plasma membrane of the cell (e.g. immune cell, e.g. Treg). This will generally be positioned next to or close to the ligand binding domain, generally upstream of the ligand binding domain, at the end of the chimeric receptor molecule/construct.

It can thus be seen that the chimeric receptor may comprise an exodomain comprising the ligand binding domain and signal sequence, if present, linked via an optional hinge domain to a transmembrane domain. The transmembrane domain may be linked to an endodomain which comprises one or more signalling domains, or alternatively or additionally, the endodomain may be provided within a second polypeptide which associates with the first polypeptide (which comprises the exodomain comprising the ligand binding domain and signal sequence, if present, linked via an optional hinge domain to a transmembrane domain). In one aspect, the transmembrane and endodomains, may be viewed as a “signalling tail” in the CAR construct. The order of the domains in a single polypeptide CAR construct is thus, N-terminal to C-terminal: exodomain-optional hinge domain-transmembrane domain-endo domain. Within the exodomain and endodomain the separate domains may be arranged in any order. Preferably however the order is signal sequence-ligand binding domain (-hinge domain, if present) in the exodomain. In one embodiment, in the endodomain the order may be co-stimulatory domain(s)-intracellular signalling domain(s). In another embodiment, the order may be intracellular signalling domain(s)-co-stimulatory domain(s). Within a multichain CAR construct, the order of domains may be, N terminal to C-terminal in a first polypeptide chain—exodomain-optional hinge domain-transmembrane domain-optional endodomain, and may be, N terminal to C-terminal in a second polypeptide chain—optional transmembrane domain-endodomain. Within the exodomain and endodomain the separate domains may be arranged in any order. Preferably however the order is signal sequence-ligand binding domain (-hinge domain, if present) in the exodomain. In one embodiment, in the endodomain the order may be co-stimulatory domain(s)-intracellular signalling domain(s). In another embodiment, the order may be intracellular signalling domain(s)-co-stimulatory domain(s). As discussed in detail below, in a multichain chimeric receptor of the invention, it is possible for one polypeptide chain to comprise an intracellular signalling sequence within its endodomain, and for the other polypeptide chain to not comprise an endodomain or to comprise an endodomain without an intracellular signalling domain. Alternatively, both polypeptide chains may comprise an endodomain comprising an intracellular signalling domain. Similarly, it is possible for one polypeptide chain to comprise at least one costimulatory domain within its endodomain (with or without an intracellular signalling domain). Both chains may comprise at least one costimulatory domain.

As noted above, the chimeric receptor, and more particularly the exodomain thereof, may also comprise a signal sequence (or targeting domain). Such a sequence will generally be provided at the N-terminal end of the molecule (construct) and may function to, co-translationally or post-translationally, direct transfer of the molecule. In particular, the signal sequence may be a sequence that targets the chimeric receptor to the plasma membrane of the immune cell (e.g. Treg). In some embodiments, the signal sequence is a CD8a signal sequence (e.g. SEQ ID NO: 9). In some embodiments, the signal sequence is the TREM2 signal sequence (e.g. SEQ ID NO: 10). This may be linked directly or indirectly (e.g. via a linker sequence) to the ligand binding domain, generally upstream of the ligand binding domain, at the N-terminal end of the chimeric receptor molecule/construct.

The linker sequence may be between 1-30, more preferably 1-25, 1-22 or 1-20, amino acids long. The linker may be a flexible linker. Suitable linkers can be readily selected and can be of any of a suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids or longer.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the chimeric receptors described herein. In one embodiment the signal sequence is linked directly to the N-terminal end of the ligand binding domain, e.g. to the N-terminal end of ligand binding domain of TREM2 or a functional variant thereof.

The ligand binding domain of the chimeric receptor is optionally followed by a hinge domain. The hinge region in a chimeric receptor is generally between the transmembrane domain and the ligand binding domain. In certain embodiments, a hinge region is an immunoglobulin hinge region (e.g. derived from IgG1, IgG2 or IgG4) and may be a wild-type immunoglobulin hinge region or an altered wild type immunoglobulin hinge region, for example a truncated hinge region. In some embodiments, the hinge region may be derived from the extracellular regions of type 1 membrane proteins such as CD8a, CD4, CD28 and CD7, which may be wild-type hinge regions from these molecules or may be altered. In some embodiments, the exodomain comprises a portion and/or variant stalk region of the TREM2 exodomain as defined above that does not contain a dipeptide sheddase cleavage site (e.g. the cleavage site has been deleted or mutated as defined above or the stalk domain has been modified to confer resistance to a sheddase enzyme). Thus, in some embodiments, the portion or variant stalk domain of the TREM2 exodomain may be viewed as the hinge domain of the chimeric receptor. Alternatively viewed, the hinge domain may be derived from the exodomain of TREM2, e.g. from the stalk domain.

Thus, in some embodiments, the hinge region is, or is derived from, the hinge region of human CD8a, CD4, CD28, CD7 or TREM2. The hinge region is alternatively (and interchangeably) referred to as a spacer or spacer region.

An “altered wild type hinge region” or “altered hinge region” or “altered spacer” refers to (a) a wild type hinge region with up to 30% amino acid changes (e.g. up to 25%, 20%, 15%, 10%, or 5% amino acid changes e.g. substitutions or deletions), (b) a portion of a wild type hinge region that is at least 10 amino acids (e.g., at least 12, 13, 14 or 15 amino acids) in length with up to 30% amino acid changes (e.g., up to 25%, 20%, 15%, 10%, or 5% amino acid changes, e.g. substitutions or deletions), or (c) a portion of a wild type hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). When an altered wild type hinge region is interposed between and connecting the TREM2 ligand binding domain and another region (e.g., a transmembrane domain) in the chimeric receptors described herein, it allows the chimeric receptor to maintain specific binding to a TREM2 ligand, e.g. ApoE, cell debris or dead or dying cells.

In certain embodiments, one or more cysteine residues in a wild type immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Hinge regions comprising the CH2 and CH3 constant region domains are described in the art for use in chimeric receptors (for example the CH2CH3 hinge, referred to as as an “Fc hinge” or “IgG hinge”). However, it is preferred that when the hinge domain is based on or derived from an immunoglobulin it does not comprise a CH3 domain, e.g. it may comprise or consist of the CH2 domain or a fragment or part thereof, without including CH3.

In one preferred embodiment the hinge domain has or comprises the amino acid sequence of SEQ ID NO: 11 (which represents the hinge domain of CD28) or an amino acid sequence having at least 95% sequence identity thereto.

The transmembrane domain of a chimeric receptor of the invention may be based on or derived from the transmembrane domain of any transmembrane protein. Typically it may be, or may be derived from, a transmembrane domain from a protein selected from a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CDS, CD22, CD79a, CD79b, CD66d, TNF-alpha, NF-kappaB, a TLR (toll-like receptor), TLRS, Myd88, lymphocyte receptor chain, IL-2 receptor, IgE, IgG, CD16a, FcγRlll, FcγRll, CD28, 4-1BB, CD4, CD8, e.g. CD8a, NKG2D (CD314) and TREM2, preferably from a human said protein. In one embodiment, the transmembrane domain may be, or may be derived from, a transmembrane domain from CD8a, CD28, CD4, CD3, NKG2D or TREM2 preferably from human said protein. In another embodiment the transmembrane domain may be synthetic in which case it would comprise predominantly hydrophobic residues such as leucine and valine.

In a preferred embodiment the transmembrane domain is the CD28 transmembrane domain having the amino acid sequence of SEQ ID NO. 12 or an amino acid sequence having at least 95% sequence identity thereto.

In some embodiments, the transmembrane domain is, or may be derived from a transmembrane domain of a protein that heterodimerises with DAP10 or DAP12 (DNAX-activating protein 10 or 12). For instance, NKG2D (CD314) dimerises with DAP10 and the TREM2 protein dimerises with DAP12. Thus, in some embodiments, the transmembrane domain is, or may be derived from, a transmembrane domain of NKG2D or TREM2.

The “endodomain” is the domain of the chimeric receptor which is present inside or within a cell when expressed therein. The chimeric receptor of the invention comprises an endodomain which comprises an intracellular signalling domain. In a single chain chimeric receptor of the invention there is typically one endodomain which comprises an intracellular signalling domain and which may additionally comprise at least one costimulatory domain, as discussed below. In a multichain chimeric receptor of the invention, for example, comprising two polypeptide chains, at least one of the chains comprises an endodomain comprising an intracellular signalling domain. In one embodiment, both chains may comprise an endodomain, and therefore a chimeric receptor may comprise two endodomains. It will be appreciated that whilst only one endodomain needs to comprise an intracellular signalling domain, it is possible for both chains to comprise this domain, which may be the same or different. Any endodomain present within a multichain chimeric receptor of the invention may comprise one or costimulatory domains.

The “intracellular signalling domain” refers to the part of the chimeric receptor that participates in transducing the message of effective chimeric receptor binding to a target antigen (i.e. TREM2 ligand) into the interior of a particular cell, e.g. an immune cell to elicit cell function, e.g. activation, cytokine production, proliferation or other cellular responses elicited with ligand binding to the exodomain of the chimeric receptor. The intracellular signalling domain is comprised within the endodomain of the receptor. The term “cell function” refers to a specialized function of a cell. Cell function of a T cell, for example, may be cytolytic activity or help, activity including the secretion of a cytokine or immunosuppressive activity. Thus, the term “intracellular signalling domain” refers to the portion of a protein that transduces a function signal and that directs a cell to perform a specialized function. While the entire intracellular signalling domain of a protein can be employed, in many cases it is not necessary to use the entire domain. To the extent that a truncated portion of an intracellular signalling domain is used, such truncated portion may be used in place of the entire domain as long as it transduces the effector function signal. The term intracellular signalling domain therefore includes any truncated portion of an intracellular signalling domain sufficient to transduce a function signal. The intracellular signalling domain is also known as the, “signal transduction domain,” and is typically derived from portions of the human CD3 or FcRy chains. Alternatively, or additionally, the signal transduction domain may be derived from DAP12/DAP10. It will be appreciated that the chimeric receptors of the invention may be introduced into cells, such as precursor or progenitor cells where the intracellular signalling domain may not be capable of inducing a cell function. However, the chimeric receptor should be capable of signalling once expressed within an appropriate cell type, e.g. an immune cell, e.g. a T cell.

It will be appreciated by a skilled person that intracellular signalling domains which comprise more than one ITAM sequence may be modified to optimise the number of ITAM sequences within the chimeric receptor. Thus, intracellular signalling domains may be modified to comprise fewer ITAM sequences, e.g. to delete one or more of the ITAM sequences present, or to modify or mutate (e.g. substitute) the amino acid residues which constitute one or more of the ITAM sequences to prevent their signalling. Preferably, at least one or two functional ITAM sequence may be retained within an intracellular signalling domain.

In a particular embodiment, an intracellular signalling domain of CD3zeta or a portion thereof may be modified to delete or to substitute one or two of the ITAM sequences. The signalling portion of CD3zeta comprises three ITAM sequences and in one embodiment, it may be desirable to utilise CD3zeta or a portion thereof which only comprises one or two functional ITAMs, particularly when CD3zeta or a portion thereof is used in direct combination with another intracellular signalling domain, e.g. with an intracellular signalling domain from DAP10 or DAP12.

Additionally, to allow or to augment full activation of the cell, e.g. the immune cell, the chimeric receptor may be provided with one or more secondary or co-stimulatory domains within an endodomain of the chimeric receptor. Thus, the intracellular signalling domain may initiate ligand dependent primary activation (i.e. may be a primary cytoplasmic signalling sequence) and the co-stimulatory domain may act in a ligand independent manner to provide a secondary or co-stimulatory signal (secondary cytoplasmic signalling sequence(s)). Primary cytoplasmic signalling sequences may regulate primary activation, including in an inhibitory way. Primary cytoplasmic signalling sequences that act in a co-stimulatory manner may contain signalling motifs which are known as immunoreceptor tyrosine-based activation motif or ITAMs.

In some embodiments, the primary signaling domain is a signaling domain from a protein selected from a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CDS, CD22, CD79a, CD79b, CD66d, TNF-alpha, NF-KappaB, a TLR (toll-like receptor), TLRS, Myd88, TOR/CD3 complex, lymphocyte receptor chain, IL-2 receptor, IgE, IgG, CD16α, FcγRIII, FcγCD28, 4-1BB, and any combination thereof. In some embodiments, the signaling domain is a signaling domain selected from a 4-1BB intracellular domain, a CD3-zeta ITAM domain, a CD3-zeta intracellular domain, a CSF-1R receptor tyrosine kinase (RTK) intracellular domain, a DAP12 intracellular domain, a TCR-zeta intracellular domain, a TL5 intracellular domain, a CD28 intracellular domain, a DAP10 intracellular domain, an FcR-gamma intracellular domain, and any combination thereof.

Examples of ITAM containing primary cytoplasmic signaling sequences that may be used in the invention include those derived from TCRζ, FcRγ, FcRβ, CD3γ, CD3δ, CD3ϵ, CDS, CD22, CD79a, CD79b and CD66d. In some embodiments, the intracellular signaling domain is derived from CD3ζ or FcRγ, preferably human CD3ζ or FcRγ.

In a preferred representative embodiment, an intracellular signalling domain as used in the invention is preferably a human CD3ζ domain, more preferably a human CD3ζ domain having the amino acid sequence of SEQ ID NO: 13 or an amino acid sequence having at least 95% sequence identity thereto.

The term “co-stimulatory signalling domain” or “co-stimulatory domain”, refers to the portion of the chimeric receptor comprising the intracellular domain of a co-stimulatory molecule. Co-stimulatory molecules are cell surface molecules other than antigen receptors or Fc receptors that provide a second signal that is typically required for efficient activation and function of an immune cell (e.g. a T-cell) upon binding to antigen. Examples of such co-stimulatory molecules include CD27, CD28, 4-IBB (CD137), OX40 (CD134), CD30, CD40, ICOS (CD278), LFA-1, CD2, CD7, LIGHT, NKD2C, B7-H2 and a ligand that specifically binds CD83, more particularly the intracellular domains of such molecules. Preferably the molecules are human. Accordingly, in some preferred embodiments, co-stimulatory domain is derived from derived from 4-1BB, CD28 or OX40 (CD134), although other co-stimulatory domains are contemplated for use with the chimeric receptors described herein. The co-stimulatory domains may be used singly or in combination (i.e. one or more co-stimulatory domains may be included). The inclusion of one or more co-stimulatory signalling domains may enhance the efficacy and expansion of immune cells expressing the chimeric receptor.

In this respect, as described in WO2020/044055 (incorporated herein by reference in its entirety), it may be advantageous to include a domain in an endodomain of the chimeric receptor that comprises a STAT5 association motif and a JAK1- and/or a JAK2-binding motif. This may be particularly helpful when the chimeric receptor is expressed in a T cell (e.g. a Treg) as such receptors address the problem associated with the high IL-2 dependence of adoptively transferred Tregs without requiring exogenous IL-2 to be administered and by providing a productive IL-2 signal in an antigen-specific manner.

“Signal Transducer and Activator of Transcription 5” (STAT5) is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation results in STAT5 phosphorylation by activating the Jak1/Jak2 and Jak3 kinases via specific signalling domains present in the IL-2R13 and IL-2Ry chain, respectively. Although Jak1 (or Jak2) can phosphorylate STAT5 without the need of Jak3, STAT5 activity is increased by the transphosphorylation of both Jak1/Jak2 and Jak3, which stabilizes their activity.

“STAT5 association motif” as used herein refers to an amino acid motif which comprises a tyrosine and is capable of binding a STAT5 polypeptide. Any method known in the art for determining protein:protein interactions may be used to determine whether an association motif is capable of binding to STAT5. For example, co-immunoprecipitation followed by western blot.

Suitably, a CAR endodomain as used in the invention may comprise two or more STAT5 association motifs as defined herein. For example, a CAR endodomain used in the invention may comprise two, three, four, five or more STAT5 association motifs as defined herein. Preferably, a CAR endodomain may comprise two or three STAT5 association motifs as defined herein.

Suitably, the STAT5 association motif may exist endogenously in a cytoplasmic domain of a transmembrane protein. For example, the STAT5 association motif may be from an interleukin receptor (IL) receptor endodomain or a hormone receptor.

A CAR endodomain used in the invention may comprise an amino acid sequence selected from any chain of the interleukin receptors where STAT5 is a downstream component, for example, the cytoplasmic domain comprising amino acid numbers 266 to 551 of IL-2 receptor β chain (NCBI REFSEQ: NP_000869.1, SEQ ID NO: 14), amino acid numbers 265 to 459 of IL-7R α chain (NCBI REFSEQ: NP_002176.2, SEQ ID NO: 15), amino acid numbers 292 to 521 of IL-9R chain (NCBI REFSEQ: NP_002177.2, SEQ ID NO: 16), amino acid numbers 257 to 825 of IL-4R α chain (NCBI REFSEQ: NPJDO0409.1, SEQ ID NO: 17), amino acid numbers 461 to 897 of IL-3Rβ chain (NCBI REFSEQ: NP_000386.1, SEQ ID NO: 18), amino acid numbers 314 to 502 of IL-17 β chain (NCBI REFSEQ: NP_061195.2, SEQ ID NO: 19) or a truncated form of IL-7R α chain (SEQ ID NO:20) may be used. The entire region of the cytoplasmic domain of interleukin receptor chain may be used.

A CAR endodomain used in the invention may comprise a STATS association motif that comprises an amino acid sequence shown as SEQ ID NO: 14-20, or a variant which is at least 80, 85, 90, 95, 96, 97, 98 or 99% identical to SEQ ID NO: 14-20. For example, the variant may be capable of binding STAT5 to at least 10%, at least 20%, at least 30%, at least 40%, at least 50%, at least 60%, at least 70%, at least 80%, or at least 90% of the level of an amino acid sequence shown as one of SEQ ID NO: 14-20. The variant or derivative may be capable of binding STAT5 to a similar or the same level as one of SEQ ID NO: 14-20 or may be capable of binding STAT5 to a greater level than an amino acid sequence shown as one of SEQ ID NO: 14-20 (e.g. increased by at least 10%, at least 20%, at least 30%, at least 40% or at least 50%).

For example, the STAT5 association motif may be from IL2Rβ, IL7Rα, IL-3Rβ (CSF2RB), IL-9R, IL-17Rβ, erythropoietin receptor, thrombopoietin receptor, growth hormone receptor and prolactin receptor.

“JAK1- and/or a JAK2-binding motif” as used herein refers to BOX motif which allows for tyrosine kinase JAK1 and/or JAK2 association. Suitable JAK1- and JAK2-binding motifs are described, for example, by Ferrao & Lupardus (Frontiers in Endocrinology; 2017; 8(71); which is incorporated herein by reference).

The JAK1 and/or JAK2-binding motif may occur endogenously in a cytoplasmic domain of a transmembrane protein.

For example, the JAK1 and/or JAK2-binding motif may be from Interferon lambda receptor 1 (IFNLR1), Interferon alpha receptor 1 (IFNAR), Interferon gamma receptor 1 (IFNGR1), MORA, IL20RA, IL22RA, Interferon gamma receptor 2 (IFNGR2) or IL1ORB.

The JAK1-binding motif may comprise an amino acid motif shown as SEQ ID NO: 21-27 or a variant therefore which is capable of binding JAK1.

The variant of SEQ ID NO: 21-27 may comprise one, two or three amino acid differences compared to any of SEQ ID NO: 21-27 and retain the ability to bind JAK1.

The variant may be at least 80, 85, 90, 95, 96, 97, 98 or 99% identical to any one of SEQ ID NO: 21-27 and retain the ability to bind JAK1.

In a preferred embodiment, the JAK1-binding domain comprises SEQ ID NO: 21 or a variant thereof which is capable of binding JAK1.

For example, the variant may be capable of binding JAK1 to at least 10%, at least 20%, at least 30%, at least 40%, at least 50%, at least 60%, at least 70%, at least 80%, or at least 90% of the level of a corresponding, reference sequence. The variant or derivative may be capable of binding JAK1 to a similar or the same level as a corresponding, reference sequence or may be capable of binding JAK1 to a greater level than a corresponding, reference sequence (e.g. increased by at least 10%, at least 20%, at least 30%, at least 40% or at least 50%).

The JAK2-binding motif may comprise an amino acid motif shown as SEQ ID NO: 28-30 or a variant therefore which is capable of binding JAK2.

The variant of SEQ ID NO: 28-30 may comprise one, two or three amino acid differences compared to any of SEQ ID NO: 28-30 and retain the ability to bind JAK2.

For example, the variant may be capable of binding JAK2 to at least 10%, at least 20%, at least 30%, at least 40%, at least 50%, at least 60%, at least 70%, at least 80%, or at least 90% of the level of a corresponding, reference sequence. The variant or derivative may be capable of binding JAK2 to a similar or the same level as a corresponding, reference sequence or may be capable of binding JAK2 to a greater level than a corresponding, reference sequence (e.g. increased by at least 10%, at least 20%, at least 30%, at least 40% or at least 50%).

Any method known in the art for determining protein:protein interactions may be used to determine whether a JAK1- or JAK2-binding motif is capable of binding to a JAK1 or JAK2. For example, co-immunoprecipitation followed by western blot.

An endodomain of the chimeric receptor of the invention may further comprise a JAK3-binding motif.

“JAK3-binding motif” as used herein refers to BOX motif which allows for tyrosine kinase JAK3. Suitable JAK3-binding motifs are described, for example, by Ferrao & Lupardus (Frontiers in Endocrinology; 2017; 8(71); which is incorporated herein by reference).

Any method known in the art for determining protein:protein interactions may be used to determine whether a motif is capable of binding to JAK3. For example, co-immunoprecipitation followed by western blot.

The JAK3-binding motif may occur endogenously in a cytoplasmic domain of a transmembrane protein.

For example, the JAK3-binding motif may be from an IL-2Rγ polypeptide.

The JAK3-binding motif may comprise an amino acid motif shown as SEQ ID NO: 38 or SEQ ID NO: 39 or a variant therefore which is capable of binding JAK3.

The variant may be at least 80, 85, 90, 95, 96, 97, 98 or 99% identical to SEQ ID NO: 38 or SEQ ID NO: 39.

In a preferred embodiment, the CAR endodomain comprises one or more JAK1-binding domains and at least one JAK3-binding domain.

In some embodiments, an endodomain of the chimeric receptor may contain a transmembrane domain or a portion thereof of a receptor that heterodimerises with DAP10 and/or DAP12. Thus, in some embodiments, a transmembrane domain of the chimeric receptor comprises a transmembrane domain or portion thereof from NKG2D (CD314) or TREM2.

As NKG2D (CD314) is a type II transmembrane protein, it may be necessary to provide domains from this protein (e.g. transmembrane and/or signalling domains) in their reverse orientation in the chimeric receptor of the invention.

In a particular embodiment, where the chimeric receptor of the invention comprises a domain or portion that heterodimerises with DAP10 and/or DAP12, it will be appreciated by a skilled person that signalling into a cell via DAP10 and/or DAP12 (once chimeric receptor has bound to its ligand) may occur via any endogenous DAP10 and/or DAP12 which may expressed by the cell, or via the expression of exogenous DAP10 and/or DAP12 in the cell. Particularly, it will be appreciated that a chimeric receptor of the invention may signal via DAP12 and/or DAP10, even though the chimeric receptor may not comprise DAP12 and/or DAP10 or a portion thereof. Thus, the vectors may beterodimerise with DAP12/DAP10 or a portion or variant thereof wherein the DAP12/DAP10 is present or expressed as a separate polypeptide. Where exogenous DAP12/DAP10 is expressed within a cell, the DAP12/DAP10 may be expressed from the same or different vectors to the chimeric receptor of the invention. Thus, vectors of the present invention, as discussed further below, may additionally encode DAP10 and/DAP12 or a functional variant thereof (e.g. a variant which has at least 50, 60, 70, 80, 90 or 95% of the signal function of wildtype DAP10 and/or DAP12).

The introduction of exogenous DAP10 or DAP12 into a cell in combination with a single chimeric receptor polypeptide of the invention may be viewed as a multichain chimeric receptor system. In a particular embodiment, a first polypeptide chain may be provided comprising an extracellular domain as described herein, a transmembrane domain (e.g. from TREM2), and optionally an endodomain which may comprise at least one costimulatory domain and/or at least one intracellular signalling domain, together with a second polypeptide comprising DAP10 or DAP12 (or a portion thereof), which may optionally further comprise at least one costimulatory domain and/or at least one intracellular signalling domain. Multiple different combinations of costimulatory and intracellular signalling domains may be used in this embodiment. For example, the second polypeptide may comprise of consist of the transmembrane and cytoplasmic domains of DAP10 or DAP12, optionally modified with at least one costimulatory domain (e.g. CD28) and/or at least one intracellular signalling domain (e.g. derived from CD3zeta), or may comprise or consist of the transmembrane domain of DAP 10 or DAP12, in combination with at least one costimulatory domain (e.g. CD28) and/or at least one heterologous intracellular signalling domain (e.g. derived from CD3zeta). The second polypeptide may therefore be a chimeric polypeptide comprising domains derived from different proteins.

However, the following combinations are particularly preferred:
1. First polypeptide comprising an endodomain comprising a costimulatory domain (e.g. from CD28) and the second polypeptide comprising an endodomain comprising an intracellular signalling domain (e.g. from DAP10 or DAP12).
2. First polypeptide comprising an endodomain comprising an intracellular signalling domain (e.g. from CD3zeta) and the second polypeptide comprising an endodomain comprising a costimulatory domain (e.g. from CD28) and an intracellular signalling domain (e.g. from DAP10 or DAP12).
3. First polypeptide comprising an endodomain comprising a costimulatory domain (e.g. from CD28) and the second polypeptide comprising an endodomain comprising an intracellular signalling domain from CD3zeta and an intracellular signalling domain (e.g. from DAP10 or DAP12).
4. First polypeptide not comprising an endodomain comprising a costimulatory domain or an intracellular signalling domain and the second polypeptide comprising a costimulatory domain (e.g. from CD28), an intracellular signalling domain, e.g. from CD3zeta and an intracellular signalling domain (e.g. from DAP10 or DAP12).

In a preferred embodiment of a chimeric receptor of the invention, the co-stimulatory domain may be, or may include, the intracellular domain of human CD28. Thus, in some embodiments, the co-stimulatory domain comprises a domain having the amino acid sequence of SEQ ID NO. 31 or an amino acid sequence having at least 95% sequence identity thereto.

The domains present within an endodomain, e.g. the intracellular signalling and one or more co-stimulatory signalling domains, may be linked in any order in tandem to the carboxyl terminus of a transmembrane domain. The domains may be linked to the carboxyl terminus of a transmembrane domain directly or indirectly, e.g. via a linker or hinge domain as described elsewhere herein.

In a preferred embodiment of the invention, the chimeric receptor comprises an optional signal peptide from CD8α; amino acid residues 19-130 of SEQ ID NO. 2 (the ligand binding domain of TREM2) or a functional variant thereof as defined herein; an optional hinge, transmembrane domain, co-stimulatory domain from CD28; and a CD3 intracellular signalling domain.

Thus, in some embodiments, the chimeric receptor comprises an amino acid sequence of SEQ ID NO. 32, 33 or 40 to 56, or an amino acid having at least 90% (e.g. at least 95%) sequence identity thereto, wherein the chimeric receptor has the functional properties defined herein, particularly wherein the chimeric receptor binds specifically to a TREM2 ligand, preferably ApoE. The chimeric receptor, i.e. the exodomain of the chimeric receptor, may be resistant to cleavage by a sheddase.

In a further preferred embodiment, the chimeric receptor comprises a signal peptide from CD8a; amino acid residues 19-130 of SEQ ID NO. 2 (the ligand binding domain of TREM2), an optional hinge, amino acid residues 175-195 of SEQ ID NO. 1 (the transmembrane domain of TREM2); and a CD3 intracellular signalling domain or a functional variant thereof as defined herein. The chimeric receptor, i.e. the exodomain of the chimeric receptor, may be resistant to cleavage by a sheddase.

The chimeric receptor may comprise an amino acid sequence of any one of SEQ ID Nos 40 to 61 and 69, or a sequence having at least 90% identity thereto (e.g. at least 95% identity thereto). In one embodiment, the chimeric receptor may comprise an amino add sequence of any one of SEQ ID Nos 44, 45, 46 or 48 or a sequence having at least 90% identity thereto (e.g. at least 95% identity thereto). In one embodiment, the chimeric receptor may comprise an amino acid sequence of any one of SEQ ID Nos 57, 58, 59, 60 or 61, or a sequence having at least 90% identity thereto (e.g. at least 95% identity thereto). In one embodiment, the chimeric receptor may comprise an amino acid sequence of any one of SEQ ID Nos 57, 58, 59 or 60, or a sequence having at least 90% identity thereto (e.g. at least 95% identity thereto). In one embodiment, the chimeric receptor may comprise an amino acid sequence of SEQ ID No 69, or a sequence having at least 90% identity thereto (e.g. at least 95% identity thereto). In another embodiment, the chimeric receptor may comprise an amino acid sequence of any one of SEQ ID NOs 40 to 56, or a sequence having at least 90% identity thereto (e.g. at least 95% identity thereto). The chimeric receptor, i.e. the exodomain of the chimeric receptor, may be resistant to cleavage by a sheddase.

It will be appreciated by those of ordinary skill in the art that, as a result of the degeneracy of the genetic code, there are many nucleotide sequences that may encode a chimeric receptor as described herein.

Accordingly, in a further aspect, the invention provides a nucleic acid molecule encoding a chimeric receptor as defined herein.

A nucleic acid molecule of the invention may comprise the nucleotide sequence of SEQ ID NO: 34 or 35 or a nucleotide sequence having at least 90% sequence identity thereto.

The nucleic acid, when expressed by a cell, causes the encoded polypeptide or polypeptides (i.e. chimeric receptor) to be expressed at the cell-surface of the cell.

The nucleic acid molecule may be RNA or DNA, such as cDNA.

The nucleic acid molecule may be introduced into a cell, particularly an immune cell, as mRNA or as DNA for expression in the cell. Vectors may be used to transfer the nucleic acid molecule into the cell or to produce the nucleic acid for transfer (e.g. to produce mRNA for transfer, or to produce a nucleic acid molecule for preparation of an expression vector for transfer into a cell).

Thus, in a further aspect, the invention provides a vector comprising the nucleic acid molecule of the invention.

In some embodiments, the vector is capable of transfecting or transducing a cell (e.g, Treg), such that it expresses the polypeptide or polypeptides (i.e. chimeric receptor).

The vector may be a non-viral vector such as a plasmid. Plasmids may be introduced into cells using any well-known method of the art, e.g, using calcium phosphate, liposomes, or cell penetrating peptides (e.g. amphipathic cell penetrating peptides).

The vector may be a viral vector, such as a retroviral, e.g. a lentiviral vector or a gamma retroviral vector.

Vectors suitable for delivering nucleic acids for expression in mammalian cells are well-known in the art and any such vector may be used. Vectors may comprise one or more regulatory elements, e.g. a promoter.

Delivery systems are also available in the art which do not rely on vectors to introduce a nucleic acid molecules into a cell, for example, systems based on transposons, CRISPR/TALEN delivery and mRNA delivery. Any such system can be used to deliver a nucleic acid molecule according to the present invention.

In some embodiments, it may be useful or necessary to express more than one polypeptide in the cell. In a representative embodiment, as discussed above, the chimeric receptor of the invention may comprise more than one polypeptide chain e.g. may comprise two polypeptide chains which heterodimerise with each other in the case of a multichain chimeric receptor. Further, the chimeric receptor of the invention may beterodimerise with another protein (e.g. an accessory protein) (i.e. comprise one or more domains that function to enable the chimeric receptor to heterodimerise with another protein (e,g. an accessory protein). While the other protein (e.g. accessory protein) may be present in the host cell, in some embodiments, it may be advantageous to modify the cell to express the other protein, e.g. where the cell does not endogenously produce the protein or does so at low levels.

Thus, in some embodiments, a nucleic acid molecule (e.g. a vector) encoding a DAP10 or DAP12 protein, preferably a human DAP10 or DAP12 protein, may be introduced (e.g. transfected or transduced) into a cell. As discussed previously, the DAP10 or DAP12 may additionally be linked to an additional intracellular signalling domain and/or costimulatory domain, and/or may be modified to comprise a portion of DAP10/DAP12 (e.g. a transmembrane and/or an intracellular signaling portion).

Thus, in some embodiments, the vector may comprise a nucleic acid encoding a first polypeptide and a nucleic acid encoding a second polypeptide of a multichain chimeric receptor, or a nucleic acid encoding the chimeric receptor and a nucleic acid encoding another polypeptide (e.g. an accessory protein). The vector may comprise the nucleic acid molecules as separate entities, or as a single nucleotide sequence, If they are present as a single nucleotide sequence, they may comprise one or more internal ribosome entry site (IRES) sequences or other translational coupling sequences between the two encoding portions to enable the downstream sequence to be translated. A cleavage site such as a 2A cleavage site (e.g. T2A, F2A or P2A) may be encoded by a nucleic acid. Alternatively, the nucleic acid encoding a first polypeptide and the nucleic acid encoding a second polypeptide of a multichain chimeric receptor or the nucleic acid encoding the chimeric receptor of the invention and the nucleic acid encoding another protein may be introduced to a cell as separate entities, e.g. on different vectors.

In one aspect of the invention, the vector encodes a chimeric receptor having an amino acid sequence of any one of SEQ ID Nos 40 to 61, or a sequence having at least 90% identity thereto (e.g. at least 95% identity thereto), wherein the chimeric receptor has the functional properties defined herein, particularly wherein the chimeric receptor binds specifically to a TREM2 ligand, preferably ApoE.

Other polypeptides may further be encoded by a nucleic acid or vector of the invention, for example, a polypeptide that may be capable of inducing cell lysis upon activation to provide a safety switch feature.

The present invention also provides a cell that expresses the chimeric receptor of the invention. The cell may co-express the chimeric receptor and a further polypeptide (e.g. DAP10 or DAP12) at the cell surface.

The present invention also provides a cell comprising a nucleic acid molecule or vector encoding a chimeric receptor of the invention.

The cell may be a cell into which a nucleic acid molecule or vector as described herein has been introduced. The cell may have been transduced or transfected with a vector according to the invention.

The cell may be suitable for adoptive cell therapy.

The cell may be any cell but particularly may be an immune cell or a precursor thereof. A precursor cell may also be termed a progenitor cell, and the two terms are used synonymously herein. Precursors of immune cells include pluripotent stem cells, e.g. induced PSC (iPSC), or more committed progenitors including multipotent stem cells, or cells which are committed to a lineage. Precursor cells can be induced to differentiate into immune cells in vivo or in vitro. In one aspect, a precursor cell may be a somatic cell which is capable of being transdifferentiated to an immune cell.

Most notably the cell may be an immune cell, such as an NK cell, a dendritic cell, a NKT cell, a MDSC, a neutrophil, a macrophage or a T cell, such as a cytotoxic T lymphocyte (CTL; CD8+ T cells), helper T cells (HTLs; CD4+ T cells) or a regulatory T cell (Treg cell). Memory or naive T cell populations may be used. The T cell may have an existing specificity. For example, it may be an Epstein-Barr virus (EBV)-specific T cell. Alternatively, the T cell may have a redirected specificity, for example, by introduction of an exogenous or heterologous TCR or a chimeric receptor, e.g. CAR.

In a preferred embodiment the immune cell is a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs. Antigen-specific Tconv cells co-cultured with Tregs as described herein may proliferate 5%, 10%, 20%, 30%, 40%, 50%, 60%, 70%, 90%, 95% or 99% less than the same Tconv cells cultured in the absence of Tregs as described herein.

Antigen-specific Tconv cells co-cultured with Tregs may express at least 10%, at least 20%, at least 30%, at least 40%, at least 50%, or at least 60% less effector cytokine than corresponding Tconv cells cultured in the absence of Tregs.

The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Tregs may also express CTLA-4 (cytotoxic T-lymphocyte associated molecule-4) or GITR (glucocorticoid-induced TNF receptor).

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻T cell, or a CD4⁺CD25⁺FOXP3⁺CD127^{-/low} T cell.

A Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Different subpopulations of Tregs are known to exist, including naïve Tregs (CD45RA+FoxP3^{low}), effector/memory Tregs (CD45RA⁻FoxP3^{high}) and cytokine-producing Tregs (CD45RA⁻FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45ROetc, expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a

Treg cell which expresses CD45RA (particularly which expresses CD45RA on the cell surface). Naïve Tregs are thus described as CD45RA+. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45R0⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺.

Isolated naive Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out as part of the invention, and methods for determining the presence and/or levels of expression of cell markers are well-known in the art and include, for example, flow cytometry, using commercially available antibodies.

In some embodiments, the nucleic acid molecule encoding the chimeric receptor is transferred to the cell e.g. the immune cell (e.g. Treg) using a viral vector, for example, a retroviral vector. In this way, a large number of antigen-specific cells (e.g. immune cells) can be generated for adoptive cell transfer. When the chimeric receptor binds the target-antigen (i.e. TREM2 ligand), this results in the transmission of an activating signal to the immune cell (e.g. Treg) it is expressed on. Thus, the chimeric receptor directs the specificity of the cell (e.g. immune cell) (e.g. Treg) towards cells expressing the targeted antigen. Accordingly, the cell comprising the nucleic acid molecule of the invention may be viewed as an “engineered cell”.

An “engineered cell” as used herein means a cell which has been modified to comprise or express a polynucleotide that is not naturally encoded by the cell. Methods for engineering cells are known in the art and include, but are not limited to, genetic modification of cells, e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid molecule into a cell. Non-viral technologies such as amphipathic cell penetrating peptides may be used to introduce a nucleic acid molecule in accordance with the present invention.

Accordingly, the nucleic acid molecule of the invention is not naturally expressed by a corresponding, unmodified cell. Suitably, an engineered cell is a cell which has been modified e.g. by transduction or by transfection. Suitably, an engineered cell is a cell which has been modified or whose genome has been modified, e.g. by transduction or by transfection. Suitably, an engineered cell is a cell that has been modified or whose genome has been modified by retroviral transduction. Suitably, an engineered cell is a cell which has been modified or whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells according to the present invention may be generated by introducing DNA or RNA encoding a chimeric receptor as described herein by one of many means including transduction with a viral vector, transfection with DNA or RNA. Cells may be activated and/or expanded prior to, or after, the introduction of a polynucleotide encoding the chimeric receptor as described herein, for example by treatment with an anti-CD3 monoclonal antibody or both anti-CD3 and anti-CD28 monoclonal antibodies. Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The cell (e.g. Treg) in which the chimeric receptor is to be expressed may be derived from a patient, that is from a subject to be treated. For example, the cell may have been removed from a subject and then transduced or transfected ex vivo with a vector according to the present invention to provide an engineered cell.

Alternatively, the cell may be a donor cell, for transfer to a recipient subject, or from a cell line., e.g, an NK cell line. The cell may further be a pluripotent cell (e.g. an iPSC) which may be differentiated to a desired target cell type, e.g. to a T cell, particularly to a Treg.

T cell populations which are suitable for ACT include: bulk peripheral blood mononuclear cells (PBMCs), CD8+ cells (for example, CD4-depleted PBMCs); PBMCs that are selectively depleted of T-regulatory cells (Tregs); isolated central memory (Tern) cells; EBV-specific CTLs; and tri-virus-specific CTLs and Treg cell preparations and populations as discussed above.

The present invention also comprises a cell population comprising a cell according to the present invention (e.g. an engineered Treg cell). The cell population may have been transduced with a vector according to the present invention. A proportion of the cells of the cell population may express a chimeric receptor according to the invention at the cell surface. A proportion of the cells of the cell population may co-express a chimeric receptor according to the invention and a further polypeptide (e.g. such as DAP10 or DAP12). The cell population may be ex vivo patient-derived cell population. It will be appreciated that not all cells within a cell population may express the chimeric receptor of the invention. However, in a particular embodiment, at least 50, 60, 70, 80, 90, 95 or 99% of cells express the chimeric receptor.

Adoptive transfer of genetically modified cells (e.g. immune cells) (i.e. engineered cells) such as T cells is an attractive approach for generating desirable immune responses, such as an anti-tumour immune response, or to suppress or prevent an unwanted immune response.

Thus, in a further aspect the invention provides a pharmaceutical composition comprising a cell or cell population of the invention (i.e. an engineered cell such as an engineered Treg, expressing a chimeric receptor of the invention, or a cell population comprising an engineered cell).

A pharmaceutical composition is a composition that comprises or consists of a therapeutically effective amount of a pharmaceutically active agent, i.e. the cell or cell population of the invention. It preferably includes a pharmaceutically acceptable carrier, diluent or excipient (including combinations thereof). Acceptable carriers or diluents for therapeutic use are well-known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the cell (e.g. Treg) and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The pharmaceutical composition according to the present invention may be administered in a manner appropriate for treating and/or preventing the disease described herein. The quantity and frequency of administration will be determined by such factors as the condition of the subject, and the type and severity of the subject's disease, although appropriate dosages may be determined by clinical trials. The pharmaceutical composition may be formulated accordingly.

The pharmaceutical composition of the invention or the chimeric receptor, nucleic acid, vector, cell or cell population of the invention can be administered parenterally, for example, intravenously, or they may be administered by infusion techniques. Parenteral administration as used herein means modes of administration other than enteral and topical administration, usually by injection, and includes intravenous, intramuscular, intraarterial, intrathecal, intracapsular, intraorbital, intracardiac, intraarticular, transtracheal, intradermal, intraperitoneal, subcutaneous, subcuticular, subcapsular, subarachnoid, intraspinal and intrastemal injection and infusion. The pharmaceutical composition, chimeric receptor, nucleic acid, vector, cell, or cell population of the invention may be administered intrathecally. The pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical composition may comprise cells of the invention in infusion media, for example sterile isotonic solution. The pharmaceutical composition may be enclosed in ampoules, disposable syringes or multiple dose vials made of glass or plastic.

The pharmaceutical composition may be administered in a single or in multiple doses. Particularly, the pharmaceutical composition may be administered in a single, one off dose. The pharmaceutical composition may be formulated accordingly.

The pharmaceutical composition may further comprise one or more active agents.

The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4lg), and/or drugs inhibiting specific cytokines (IL-6, IL-17, TNFalpha, IL18).

Depending upon the disease and subject to be treated, as well as the route of administration, the pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for cells (e.g. Tregs) of the invention, doses of 5×10⁷ to 3×10⁹ cells, or 10⁸ to 2×10⁹ cells per subject may be administered.

The cells may be appropriately modified for use in a pharmaceutical composition. For example, cells (e.g. Tregs) may be cryopreserved and thawed at an appropriate time, before being infused into a subject.

The invention further includes the use of kits comprising the nucleic acid, vector, cell and/or pharmaceutical composition of the present invention. Preferably said kits are for use in the methods and uses as described herein, e.g., the therapeutic methods as described herein. Preferably said kits comprise instructions for use of the kit components.

The present invention further provides a method for treating and/or preventing a disease or condition in a subject, which comprises the step of administering a cell, cell population or pharmaceutical composition according to the invention to the subject. The method may comprise the step of administering a population of cells to a subject.

The method may involve the following steps:

(i) taking a sample of cells, such as a blood sample from a patient,

(ii) extracting the immune cell, e.g. T-cells,

(iii) introducing into the cells (e.g. transducing or transfecting the cells) a vector or a nucleic acid of the present invention encoding the chimeric receptor of the invention,

(iv) expanding the cells comprising the nucleic acid or vector (i.e. the modified or engineered cells) ex-vivo, and

(v) returning the cells to the subject.

In some embodiments, steps (i) and (ii) may be viewed as providing a cell-containing sample (e.g. Treg sample), particularly obtained from a subject.

The modified (i.e. engineered) cells may possess a desired therapeutic property such as immunosuppressive activity or specific targeting and killing of target cells. It will be appreciated by the skilled person that the cells may be allogenic or autologous to the subject to be treated.

Thus, in a further aspect, the invention provides a cell, cell population or pharmaceutical composition as defined herein for use in therapy.

The cell, cell population and pharmaceutical composition of the invention may find particular utility in the treatment of disorders associated with cells that express or release a TREM2 ligand, such as ApoE, or with disorders where a TREM2 ligand is localised at or near the site of disease, particularly disorders that would benefit from the immunosuppressive activity or target killing activity of the cells of the invention. In some embodiments, the disorder is an inflammatory, allergic or autoimmune disorder (e.g. type I diabetes), particularly a neurological disorder or liver disease. An inflammatory disorder is any condition associated with unwanted inflammation or with an increase in inflammation. Inflammatory disorders include conditions such as inflammatory bowel disease. The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis and juvenile onset diabetes.

The term “target cell” refers to any cell expressing a TREM2 ligand to which the cell of the invention is to be directed to exert its therapeutic effect. In some embodiments, the target cell functions as a marker of a disease site, i.e. to attract the cells of the invention to provide an immunosuppressive effect. In some embodiments, the target cell is killed or abrogated by the cells of the invention. As noted above, in some embodiments, the target cell will be a microglial cell. In some embodiments, the target cell is a liver cell (e.g. hepatocyte). A skilled person will appreciate that a TREM2 ligand may also be secreted from a cell, and therefore that the cells of the invention may be directed to secreted proteins which are not present within or on a cell.

In some embodiments, the disease or disorder to be treated is a neurological disease or disorder/condition. In some embodiments, the neurological disease or disorder is associated with inflammation. Thus, in some embodiments, the invention may find utility in treating or preventing (e.g. reducing the risk of) neuroinflammation or an associated disease or disorder. The neuroinflammation may be chronic or acute, preferably chronic. The neuroinflammation may be neuroinflammation of the central or peripheral nervous system, preferably the central nervous system.

Thus, in a further aspect, the invention provides a method of preventing, reducing risk of, or treating a neurological disease, disorder, or injury or liver disease comprising administering to an individual in need thereof a therapeutically effective amount of the cell, cell population or pharmaceutical composition as described herein.

Alternatively viewed, the invention provides a cell, cell population or pharmaceutical composition as defined herein for use in preventing, reducing risk of, or treating a neurological disease, disorder, or injury or liver disease in an individual in need thereof.

In yet another aspect, the present invention provides the use of the cell or cell population of the invention in the manufacture of a medicament for preventing, reducing risk of, or treating a neurological disease, disorder, or injury or liver disease in an individual in need thereof.

In some embodiments, the neurological disease, disorder or injury is a neurodegenerative disease or condition, particularly associated with inflammation. Thus, in some embodiments the neurological disease, disorder or injury is selected from amyotrophic lateral sclerosis (ALS), dementia, frontotemporal dementia, Alzheimer's disease, vascular dementia, mixed dementia, Creutzfeldt-Jakob disease, Chronic Inflammatory Demyelinating Polyneuropathy (CIDP), Huntington's disease, Taupathy disease, Nasu-Hakola disease, central nervous system lupus, Parkinson's disease, dementia with Lewy bodies, Multiple System Atrophy (Shy-Drager syndrome), progressive supranuclear palsy, cortical basal ganglionic degeneration, acute disseminated encephalomyelitis, seizures, spinal cord injury, traumatic brain injury (e.g. ischemia and traumatic brain injury), depression, autism spectrum disorder and multiple sclerosis. In some embodiments, the neurological disease, disorder, or injury is amyotrophic lateral sclerosis (ALS), Alzheimer disease, Parkinson disease, multiple sclerosis, ischemia and traumatic brain injury, depression, and autism spectrum disorder.

In one embodiment, the neurological disease is amyotrophic lateral sclerosis (ALS).

Amyotrophic lateral sclerosis (ALS) (also known as motor neuron disease or Lou Gehrig's disease) refers to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

Expression of TREM2 ligands, such as ApoE, cell debris and dead or dying cells is increased in the central nervous system (CNS) of ALS patients. In addition, endogenous T cells with a regulatory phenotype home to the CNS in SOD1 mouse models of ALS. Whilst not wishing to be bound by theory, based on the data set out in the Examples the inventors hypothesize that Tregs expressing a chimeric receptor as described herein (i.e. with a TREM2 ligand binding domain) will home to, and become activated at, disease sites associated with ALS, thereby exerting the therapeutic benefits of the Tregs, e.g. promoting tissue repair and regeneration, reducing inflammation etc., at these sites. Therefore, in a specific embodiment, the present invention provides a regulatory T cell (Treg) expressing a chimeric receptor as described herein (i.e. with a TREM2 ligand binding domain) for use in treating ALS. Alternatively viewed, the invention provides a method of preventing, reducing risk of, or treating ALS comprising administering to an individual in need thereof a therapeutically effective amount of a Treg expressing a chimeric receptor as described herein.

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include frontotemporal dementia, Alzheimer's disease, vascular dementia, mixed dementia, semantic dementia, and dementia with Lewy bodies.

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments.

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain (e.g. intraneuronal accumulations of hyperphosphorylated Tau protein in neurofibrillary tangles or Pick bodies).

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

Nasu-Hakola disease (NHD), which may alternatively be referred to as polycystic lipomembranous osteodysplasia with sclerosing leukoencephalopathy (PLOSL), is a rare inherited leukodystrophy characterized by progressive presenile dementia associated with recurrent bone fractures due to polycystic osseous lesions of the lower and upper extremities. NHD disease course is generally divided into four stages: latent, osseous, early neurologic, and late neurologic. After a normal development during childhood (latent stage), NHD starts manifesting during adolescence or young adulthood (typical age of onset 20-30 years) with pain in the hands, wrists, ankles, and feet. Patients then start suffering from recurrent bone fractures due to polycystic osseous and osteroporotic lesions in the limb bones (osseous stage). During the third or fourth decade of life (early neurologic stage), patients present with pronounced personality changes (e.g., euphoria, lack of concentration, loss of judgment, and social inhibitions) characteristic of a frontal lobe syndrome. Patients also typically suffer from progressive memory disturbances. Epileptic seizures are also frequently observed. Finally (late neurologic stage), patients progress to a profound dementia, are unable to speak and move, and usually die by the age of 50.

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

Taupathy diseases, or Tauopathies, are a class of neurodegenerative disease caused by aggregation of the microtubule-associated protein tau within the brain. Alzheimer's disease (AD) is the most well-known Taupathy disease, and involves an accumulation of tau protein within neurons in the form of insoluble neurofibrillary tangles (NFTs). Other Taupathy diseases and disorders include progressive supranuclear palsy, dementia pugilistica (chromic traumatic encephalopathy), Frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, frontotemporal dementia, and frontotemporal lobar degeneration.

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

Creutzfeldt-Jakob disease (CJD) is a prion disease which has sporadic, iatrogenic, and familial forms. CJD is characterized by spongiform change (e.g., microcavitation of the brain, usually predominant in gray matter), neuronal cell loss, astrocytic proliferation disproportionate to neuronal loss, and accumulation of an abnormal amyloidogenic protein, sometimes in discrete plaques in the brain. Prions, the infectious agents that transmit these diseases differ markedly from viruses and viroids in that no chemical or physical evidence for a nucleic acid component has been reproducibly detected in infectious materials.

Central nervous system (CNS) lupus is a neurologic manifestation of systemic lupus erythematosus (SLE), a multisystem autoimmune connective tissue disorder. CNS lupus is a serious illness with neurologic symptoms which include headaches, confusion, fatigue, depression, seizures, strokes, vision problems, mood swings, and difficulty concentrating.

Multiple system atrophy (MSA), also known as Shy-Drager syndrome, is a progressive neurodegenerative disorder characterized symptoms that affect both the autonomic nervous system and movement. Symptoms are the result of progressive loss of function and death of different types of nerve cells in the brain and spinal cord and include fainting spells, heart rate problems, and bladder control. Motor impairments include tremor, rigidity, loss of muscle coordination, and difficulties with speech and gait. MSA includes disorders that historically had been referred to as Shy-Drager syndrome, olivopontocerebellar atrophy, and striatonigral degeneration. A distinguishing feature of MSA is the accumulation of the protein alpha-synuclein in glia, the cells that support nerve cells in the brain.

Progressive supranuclear palsy (PSP) is an uncommon brain disorder that results from damage to nerve cells in the brain. PSP affects movement, control of walking (gait), balance, speech, swallowing, vision, mood, behavior, thinking, and control of eye movements. The symptoms of PSP are caused by a gradual deterioration of brain cells in a few specific areas in the brain, mainly in the region called the brain stem. PSP is characterized by abnormal deposits of the protein tau in nerve cells in the brain.

Cortical basal ganglionic degeneration (CBGD) is a rare, progressive neurodegenerative disease involving the cerebral cortex and the basal ganglia. CBGD symptoms include movement and cognitive dysfunction, Parkinsonism, alient hand syndrome, and psychiatric disorders. CBGD pathology is characterized by the presence of astrocytic abnormalities within the brain and improper accumulation of the protein tau.

Acute disseminated encephalomyelitis (ADEM), or acute demyelinating encephalomyelitis, is a rare autoimmune disease characterized by widespread inflammation in the brain and spinal cord. ADEM also damages myelin insulation on nerves of the CNS, destroying the white matter. ADEM is characterized by multiple inflammatory lesions in the subcortical and central white matter and cortical gray-white junction of the cerebral hemispheres, cerebellum, brainstem, and spinal cord.

Neurological injuries can result from stroke, acute trauma, chronic trauma, seizures, spinal cord injury, traumatic brain injury (TBI), alcohol abuse, or vitamin B deficiency. Neurological injuries can result in impairment or disability, including neurocognitive deficits, delusions, speech or movement problems, intellectual disability, sleep disorders, mental fatigue, personality changes, coma or a persistent vegetative state.

Liver disease or hepatic disease refers generally to any damage or disease of the liver. In some embodiments, the liver disease is an inflammatory liver disease. In some embodiments, the liver disease is chronic liver disease,

In some embodiments, the liver disease is selected from fascioliasis, hepatitis (e.g. viral hepatitis, alcoholic hepatitis or autoimmune hepatitis), alcoholic liver disease, fatty liver disease (hepatic steatosis and steatohepatitis), hemochromatosis, Gilbert's syndrome, cirrhosis, primary binary cirrhosis and primary sclerosing cholangitis.

TREM2 is expressed in macrophages that are associated with various diseases and/or disease sites, such as fibrosis (e.g. renal fibrosis) and atherosclerosis plaques. Thus, in some embodiments, the disease or disorder to be treated is a disease or disorder/condition associated with macrophages expressing TREM2.

Fibrosis refers to conditions in which connective tissue replaces normal parenchymal tissue, resulting in tissue remodeling and the formation of scar tissue. Thus, fibrosis is also known as fibrotic scarring. Fibrosis may occur as a result of repeated injuries and/or chronic inflammation.

Fibrosis may occur in various tissues and organs, including the kidneys, lungs, liver, brain, intestines and heart. In some particular embodiments, the invention finds utility in the treatment of fibrosis of the kidney, i.e. renal fibrosis, e.g. tubulointerstitial renal fibrosis.

“Atherosclerosis” is a disease in which the inside of an artery narrows due to the build-up of plaque and can result in coronary artery disease, stroke, peripheral artery disease, or kidney problems, depending on which arteries are affected.

Thus, in a further aspect, the invention provides a method of preventing, reducing risk of, or treating fibrosis (e.g. renal fibrosis) or atherosclerosis comprising administering to an individual in need thereof a therapeutically effective amount of the cell, cell population or pharmaceutical composition as described herein.

Alternatively viewed, the invention provides a cell, cell population or pharmaceutical composition as defined herein for use in preventing, reducing risk of, or treating fibrosis (e.g. renal fibrosis) or atherosclerosis in an individual in need thereof.

In yet another aspect, the present invention provides the use of the cell or cell population of the invention in the manufacture of a medicament for preventing, reducing risk of, or treating fibrosis (e.g. renal fibrosis) or atherosclerosis in an individual in need thereof.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to affect a measurable improvement of a particular disease, disorder, or condition. A therapeutically effective amount herein may vary according to factors such as the disease state, age, sex, and weight of the patient, and the ability of the chimeric receptors to elicit a desired response in the individual. A therapeutically effective amount is also one in which any toxic or detrimental effects of the cell, cell population or pharmaceutic compositions are outweighed by the therapeutically beneficial effects.

The terms “subject”, “patient” and “individual” are used interchangeably herein and refer to a mammal, preferably a human. In particular, the terms subject, patient and individual refer to a human having a disease or disorder as defined herein in need of treatment.

In some embodiments of the invention, the patient may be subjected to other treatments prior to, contemporaneously with, or after the treatments of the present invention. For instance, in some embodiments, the patient may be treated with other procedures for the treatment of symptoms associated with the disease or disorder.

In some embodiments, the cell, cell population or pharmaceutical composition of the invention may be administered in combination with other therapeutic agents for the treatment of symptoms associated with the disease or disorder or other underlying condition.

Thus, in some embodiments, the pharmaceutical composition of the invention may contain one or more additional therapeutic agents or may be for administration with one or more additional therapeutic agents.

In some embodiments, the pharmaceutical composition may contain or be administered with a further therapeutic agent useful in treating neurological disease or liver disease.

The other therapeutic agents may be part of the same composition already comprising the cell or cell population of the invention, in the form of a mixture, wherein the cell or cell population and the other therapeutic agent are intermixed in or with the same pharmaceutically acceptable solvent and/or carrier or may be provided separately as part of a separate compositions, which may be offered separately or together in form of a kit of parts.

Thus, the cell, cell population or pharmaceutical composition of the invention may be administered concomitantly with the other therapeutic agent separately, simultaneously or sequentially. For example, the cell, cell population or pharmaceutical composition of the invention may be administered simultaneously with a first additional therapeutic agent or sequentially after or before administration of said first additional therapeutic agent. If the treatment regimen or schedule utilizes more than one additional therapeutic agent, the various agents may be partially administered simultaneously, partially sequentially in various combinations.

Thus, in some embodiments, the invention provides a cell, cell population or pharmaceutical composition of the invention in a combined product with another therapeutic agent for separate, simultaneous or sequential administration for use in treating or preventing a disease or disorder as defined herein.

Alternatively viewed, the method of the invention further comprises administering another therapeutic agent to said subject, wherein said therapeutic agent is administered separately, simultaneously or sequentially to the cell, cell population or pharmaceutical composition of the invention.

The therapeutic agents for use in combination with the cell, cell population or pharmaceutical composition of the invention may be provided in pharmaceutical compositions as defined above and may be administered as defined above. Thus, the compositions comprising additional therapeutic agents may comprise pharmaceutically acceptable excipients, solvents and diluents suitable for such formulations.

The skilled person will be aware of suitable dosage ranges for any given additional therapeutic agent. In preferred embodiments, the additional therapeutic agent is present in the pharmaceutical composition, or administered to the subject, in its typical dose range.

The invention will now be further described by way of Figures and Examples, which are meant to serve to assist one of ordinary skill in the art in carrying out the invention and are not intended in any way to limit the scope of the invention.

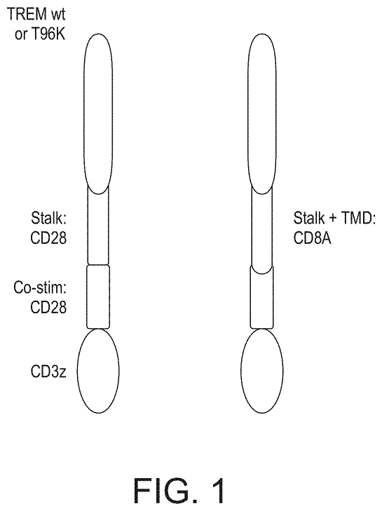

FIG. 1 shows a schematic of a single chain chimeric receptor of the invention,comprising a ligand binding domain derived from TREM2 having a wild type sequence or a mutation of T96K, together with a stalk and a transmembrane domain ™ from CD28 or from CD8 alpha, a costimulatory domain from CD28 and an intracellular signalling domain from CD3zeta.

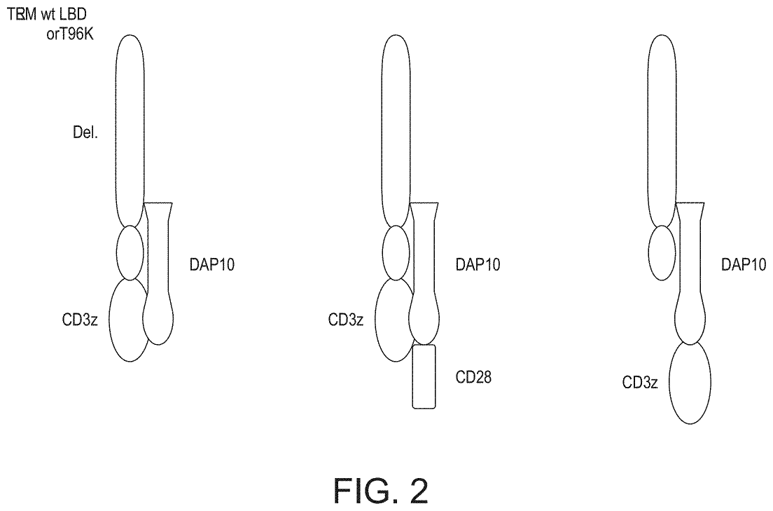

FIG. 2 shows a schematic of a multichain chimeric receptor of the invention, comprising a ligand binding domain derived from TREM2 having a wild type sequence or a mutation of T96K. The first example comprises a first polypeptide comprising a CD3zeta intracellular signalling domain and a second polypeptide comprising DAP10. The second example shows a first polypeptide comprising a CD3zeta intracellular signalling domain and a second polypeptide comprising DAP10 and a CD28 costimulatory domain. The third example show a first polypeptide with no intracellular signalling sequence or costimulatory domains, and a second polypeptide comprising DAP10 and an intracellular signalling domain of CD3zeta.

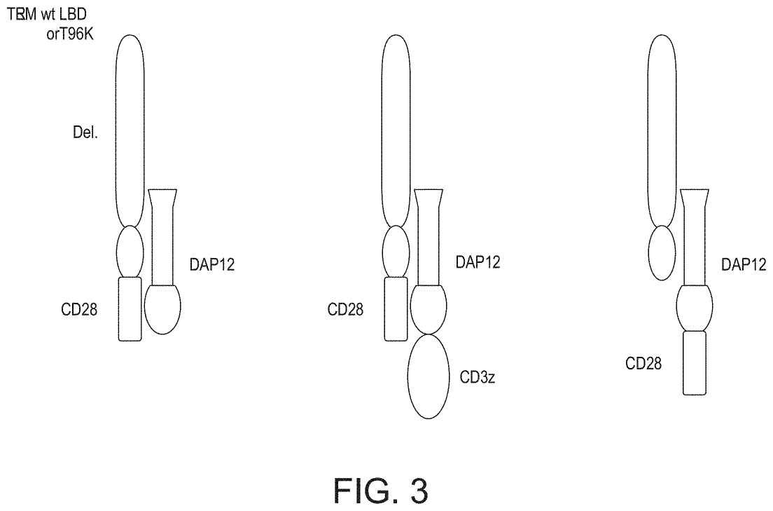

FIG. 3 shows a schematic of a multichain chimeric receptor of the invention, comprising a ligand binding domain derived from TREM2 having a wild type sequence or a mutation of T96K. The first example comprises a first polypeptide comprising a CD28 costimulatory domain and a second polypeptide comprising DAP12. The second example shows a first polypeptide comprising a CD28 costimulatory domain and a second polypeptide comprising DAP12 and a CD3zeta intracellular signalling domain. The third example show a first polypeptide with no intracellular signalling sequence or costimulatory domains, and a second polypeptide comprising DAP12 and a CD28 costimulatory domain.

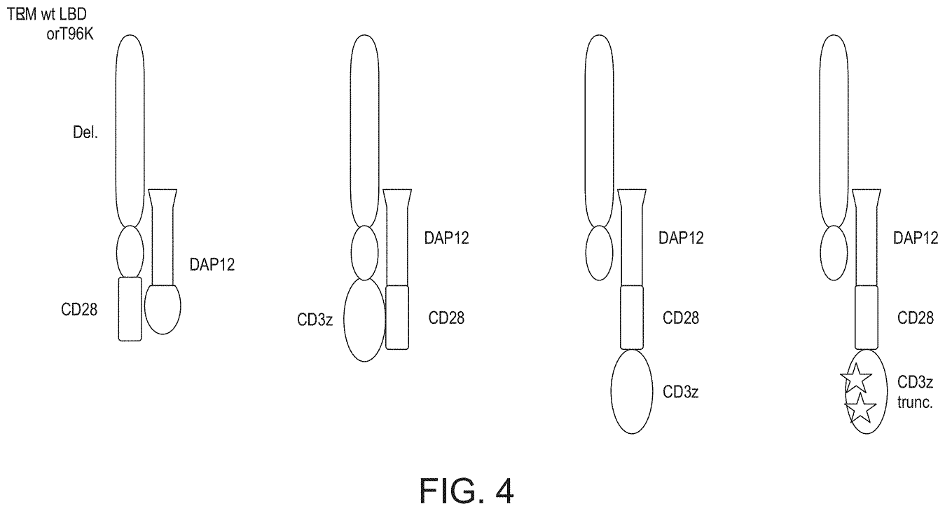

FIG. 4 shows a schematic of a multichain chimeric receptor of the invention,comprising a ligand binding domain derived from TREM2 having a wild type sequence or a mutation of T96K. The first example comprises a first polypeptide comprising a CD28 costimulatory domain and a second polypeptide comprising DAP12. The second example shows a first polypeptide comprising a CD3zeta intracellular signalling domain and a second polypeptide comprising a truncated DAP12 (transmembrane only) and a CD28 costimulatory domain. The third example shows a first polypeptide with no intracellular signalling sequence or costimulatory domains, and a second polypeptide comprising a truncated DAP12 (transmembrane only), a CD28 costimulatory domain and a CD3zeta intracellular signalling domain. The fourth example shows a first polypeptide with no intracellular signalling sequence or costimulatory domains, and a second polypeptide comprising a truncated DAP12 (transmembrane only), a CD28 costimulatory domain and a CD3zeta intracellular signaling domain, wherein one ITAM sequence has been deleted.

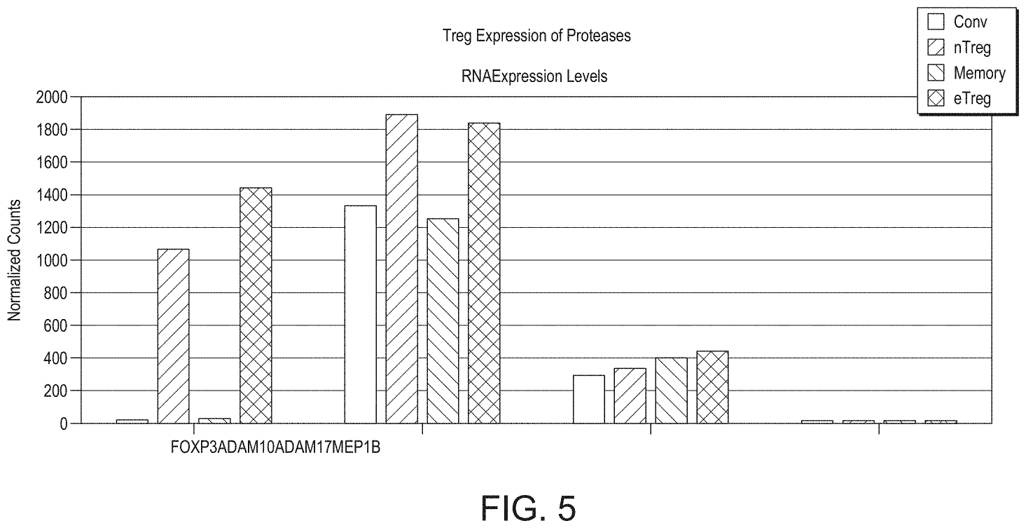

FIG. 5 shows the expression of various proteases in Tregs. Whilst ADAM10 and 17 are expressed, Meprin beta (MEP1B) is not.

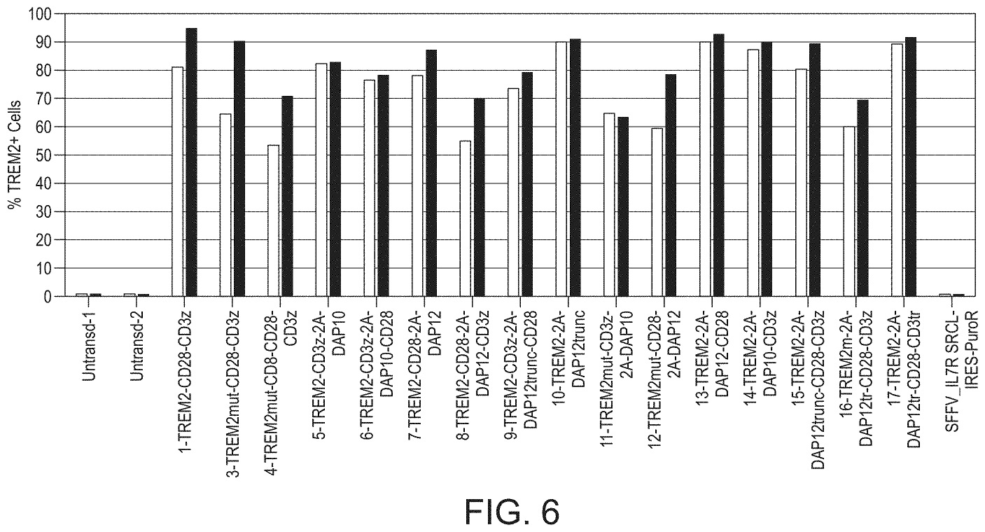

FIG. 6 shows the expression of various chimeric constructs of the invention (SEQ ID Nos 40 and 42-56) in Jurkat cells after transduction using two different concentrations of virus, using an anti-TREM2 antibody.

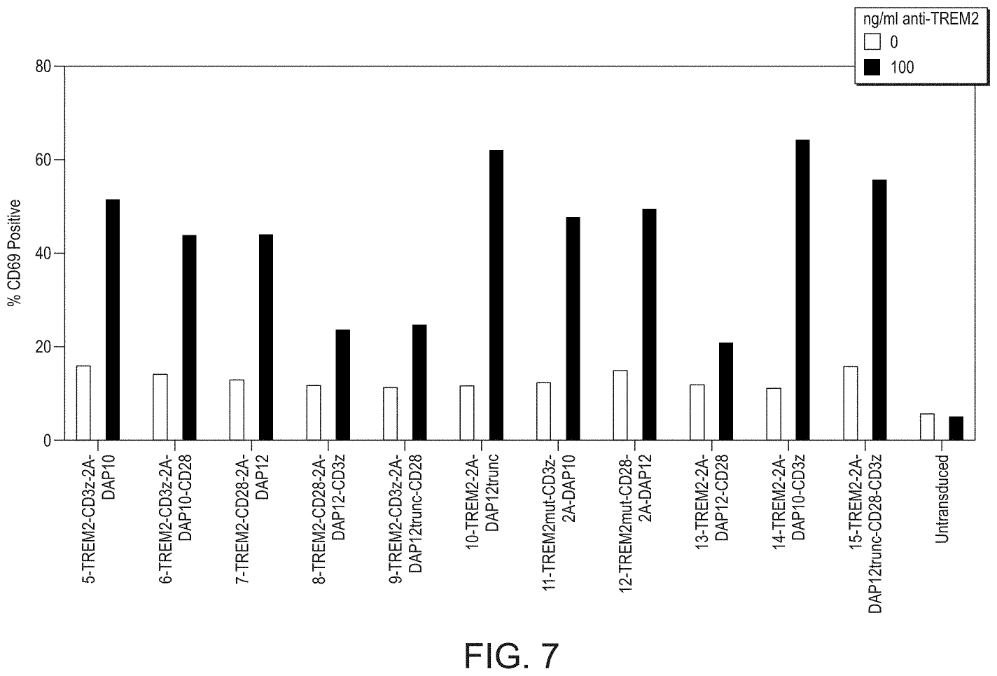

FIG. 7 shows the activation of Jurkat cells transduced with various chimeric constructs of the invention (SEQ ID Nos 44-54) using an anti-TREM2 antibody.

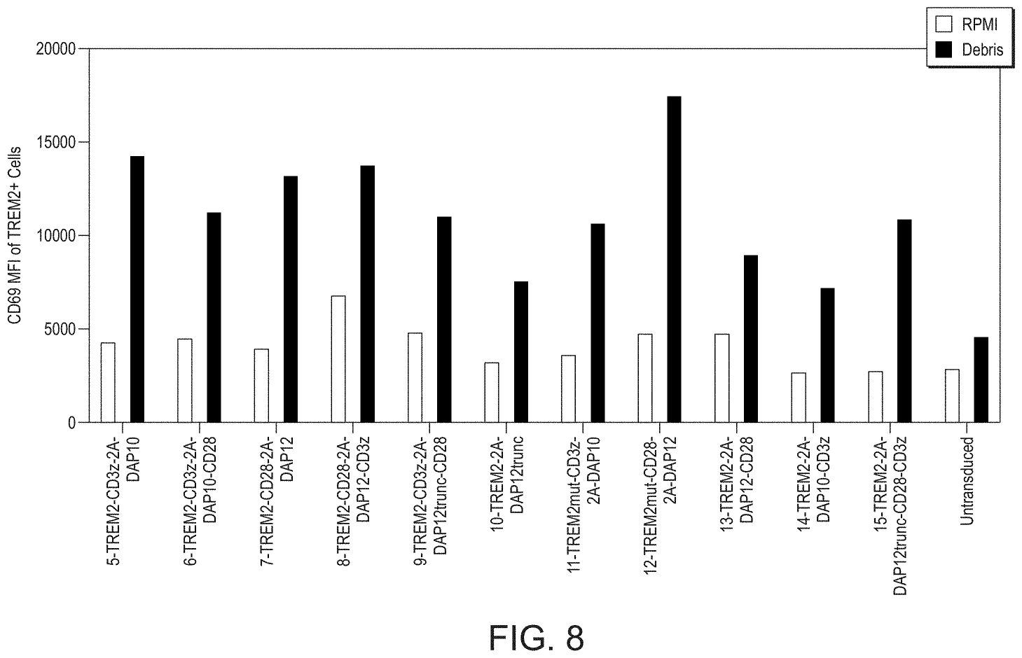

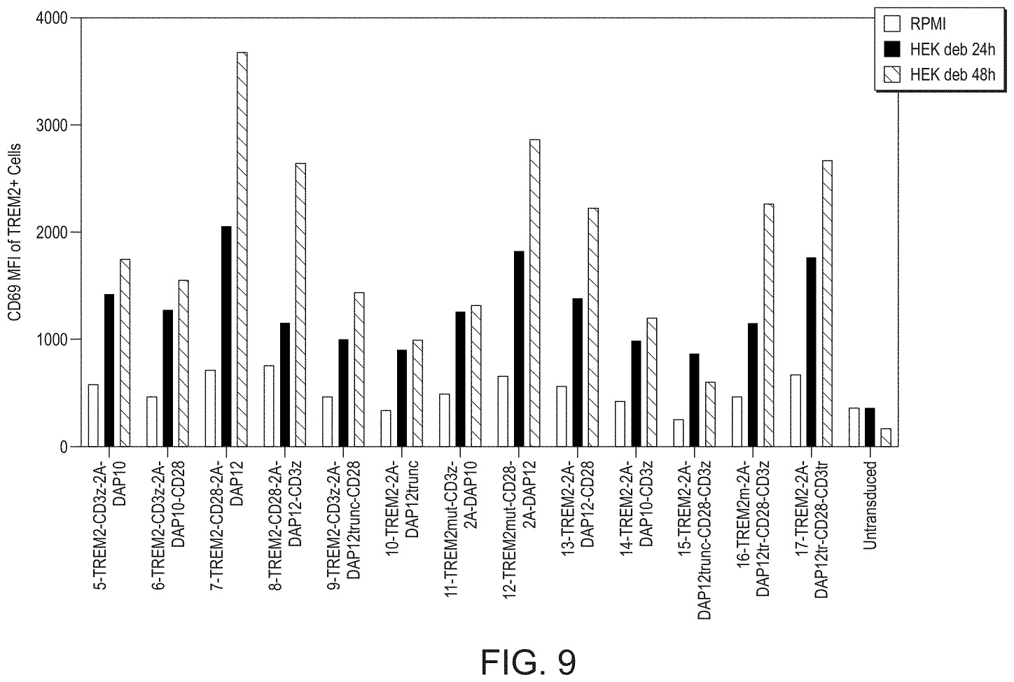

FIG. 8 shows the activation of Jurkat cells transduced with various chimeric constructs of the invention (SEQ ID Nos 44-54) using necrotic K562 cells.

FIG. 9 shows the activation of Jurkat cells transduced with various chimeric constructs of the invention (SEQ ID Nos 44-56) using HEK cell debris.

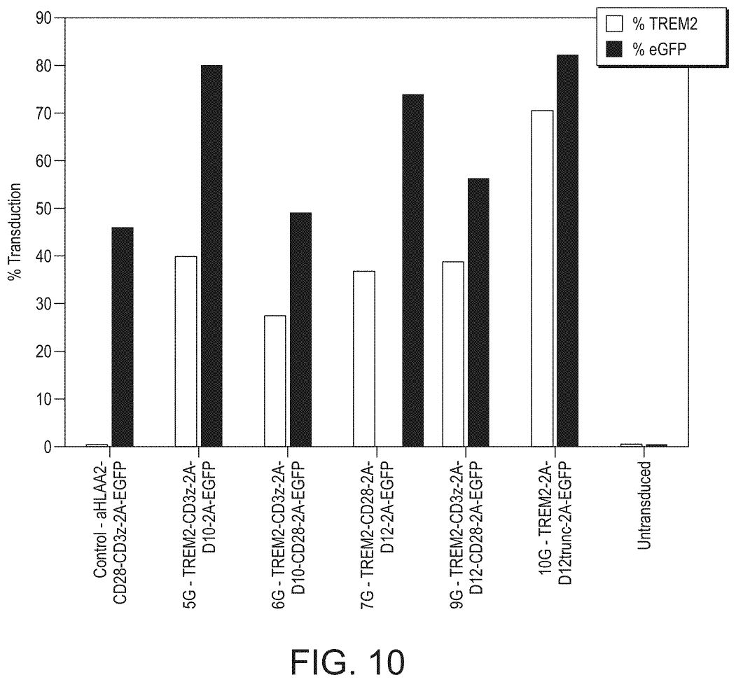

FIG. 10 shows the expression of various chimeric constructs of the invention (SEQ ID Nos 57-61) in Treg cells after transduction. SEQ ID Nos 57, 58, 59, 60 and 61 correspond to SEQ ID Nos 44, 45, 46, 48 and 49, except that they comprise an additional 2A cleavage sequence and eGFP sequence. These constructs are numbered as 5G, 6G, 7G, 9G and 10G in FIG. 10 to indicate the addition of eGFP.

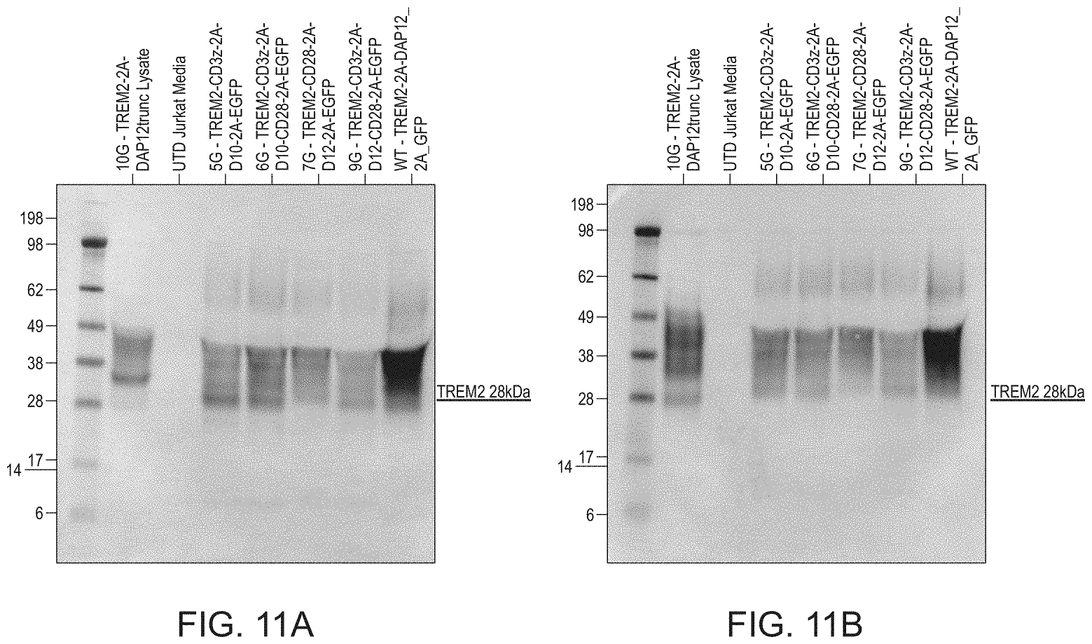

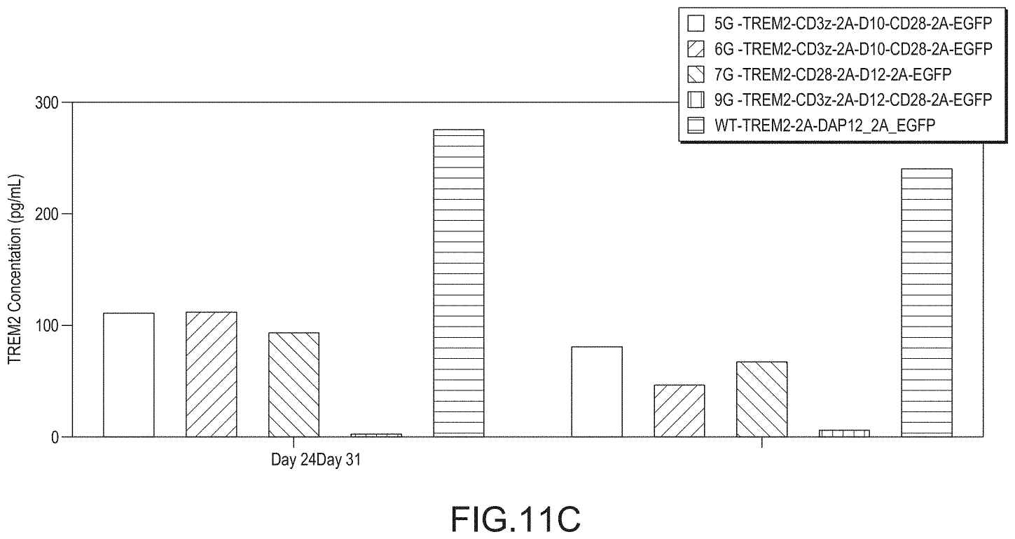

FIG. 11 shows the amount of TREM2 cleavage in various chimeric constructs of the invention comprising a mutant TREM2 exodomain (SEQ ID Nos 57-60) compared to a chimeric construct of the invention comprising the wild-type TREM2 exodomain (SEQ ID NO: 69). FIG. 11a is a Western blot of samples from cells harvested at day 11. FIG. 11b is a Western blot of samples from cells harvested at day 14. FIG. 11c is an ELISA for TREM2 using media from cells harvested at day 24 and day 31.

### EXAMPLES

### Example 1a

### Screening of TREM2 Constructs—Expression

Different constructs of the TREM2 CAR (encoding SEQ ID Nos 40 and 42-56) were cloned into a lentiviral backbone encoding a puromycin resistance gene, where the ligand binding domain of TREM2 (wildtype or mut (T96K)) was used to confer specificity to the CAR construct. Viral vectors were produced and used for the transduction of the Jurkat T cell line. Two days after transduction, Jurkat cells were selected with 4 μg/ml puromycin for one week. Cells were counted and 0.5*10⁶ cells were stained with an anti-TREM2 antibody (Human/mouse TREM2 APC-conjugated antibody from R&D systems (FAB17291A)) to determine the level of CAR expression. CAR expression was assessed by flow cytometry and can be seen in FIG. 6.

### Example 1b

### Screening of TREM2 Constructs—Activation with Antibody

Jurkat cells were transduced as described in Example 1a with constructs encoding SEQ ID Nos 44 to 54, except that the lentiviral backbone did not include a puromycin resistance gene and Jurkat cells were not selected for using puromycin. Transduced cells were activated with anti-TREM2 antibody. CAR-dependent activation levels were assessed by flow cytometry using CD69 staining and can be seen in FIG. 7,

### Example 1c

### Screening of TREM2 Constructs—Activation with Dead Cells or Cell Debris

Jurkat cells were transduced as described in Example 1a with constructs encoding either SEQ ID Nos 44-54 (FIG. 8) or SEQ ID Nos 44-56 (FIG. 9), except that the lentiviral backbone did not include a puromycin resistance gene and Jurkat cells were not selected for using puromycin. Transduced cells were either co-cultured with RPMI as control and with necrotic K562 cells to activate the cells (FIG. 8) or co-cultured with RPMI as control and with HEK cell debris for 24 or 48 hours to activate the cells. CAR-dependent activation levels were assessed by flow cytometry using CD69 staining and can be seen in FIGS. 8 and 9, respectively.

### Example 2

### NFAT/NfkB/STAT5 Signaling of CAR Constructs

Different constructs of the TREM2 CAR were cloned into a lentiviral backbone encoding a puromycin resistance gene, Viral vectors are produced and used for the transduction of a NFAT, NfkB and STAT5 Jurkat reporter cell line. Here,the NFAT, NfkB or STAT5 response element control the activity of a luc2 reporter gene. Two days after transduction, Jurkat cells are selected with 4 μg/ml puromycin for one week. Cells are then activated using ApoE2 or ApoE4 recombinant protiens coated on a cell culture plate and eight hours after activation luciferase is assessed in the different reporter cell lines using ONE-Glo™ Luciferase Assay System (Promega).

### Example 3

### Generation of Regulatory T Cells Expressing the TREM2 CAR

Regulatory T cells are purified and FACS sorted as CD4⁺ CD25⁺ CD127-cells from healthy donors. Cells are activated using Human T-Activator CD3/CD28 Dynabeads™ (ThermoFisher Scientific) in X-Vivo medium (Lonza) in the presence of Interleukin-2 (1000 IU/ml), After 48 hours of activation, cells are transduced with lentiviral particles,encoding TREM2 CAR constructs or encoding a control construct that comprises HLA-A2 instead of TREM2. All constructs also encode eGFP. Cells are further expanded, and expansion rate is compared between the different conditions. At day 14 cells are harvested and counted. 0.5*106 cells are stained with an anti-TREM2 antibody. The level of CAR expression and transduction efficiency was assessed by Flow Cytometry looking at the percentage of anti-TREM2 antibody and percentage of GFP expression, respectively. The Treg phenotype is assessed by surface staining with anti-CD4, anti-CD25, anti-CD127, anti-CD8, anti-GITR, anti-CD39, anti-CD45RA, anti-CD45RO, anti-ICOS and intracellular staining with anti-FOXP3 and anti-HELIOS, following fixation and permeabilization (Transcription Factor Staining Buffer Set, ThermoFisher Scientific)

FIG. 10 shows the expression of various TREM2 CAR constructs of the invention in regulatory T cells. Regulatory T cells were transduced with a control construct comprising HLA-A2 instead of TREM2 or with constructs comprising SEQ ID Nos 57, 58, 59, 60 and 61.

### Example 4

### Treg Suppressive Activity

For assessing the ability of Treg to suppress effector T cell activation, Teff cells are labeled with CFSE dye. Teff cells are co-cultured with different concentrations of Treg cells (ratios Treg:Teff of 1:1, 1:2, 1:4, 1:8, 1:16, 1:32, 1:64, 1:124) or no Treg cells. For activation, CD3/28 Beads (1:100) are added. For CAR-dependent activation, ApoE2 or ApoE4 recombinant proteins are added. 72 h after activation, cells are harvested and analyzed by flow cytometry. CFSE dilution is used as a surrogate marker for Teff cell proliferation.

### Example 5

### Treg Activation Assay

For analysis of CAR-dependent Treg activation, Treg are cultured in the presence of ApoE2 or ApoE4 recombinant proteins. Here, 0.1*10⁶ Treg are cultured. As a negative control, Treg are cultured in the absence of recombinant protein. As a positive control, Treg are cultured in the presence of CD3/CD28 activation beads. After 24 h cells are harvested and stained with anti-CD4, anti-CD25, anti-CD69, anti-CD137 and anti-GARP antibodies. Cells are acquired on a flow cytometer and percentage of CD69, CD137 and GARP up-regulation after stimulation are calculated.

### Example 6

### Generation of Effector CAR-T Cells

For the generation of Teff CAR-T cells, PBMCs were activated with anti-CD3 antibody (OKT3) for 48h. After activation, cells are washed and transduced with lentiviral vector encoding the TREM2 CAR. 48h after transduction, cells are washed and seeded for expansion until day 10. At day 10 cells are harvested and cryopreserved for further analysis. To assess CAR expression, cells are counted, and 0.5*106 cells are stained with TREM2 APC conjugated antibody to determine the level of CAR expression. CAR expression was assessed by flow cytometry. Cell phenotype was assessed by flow cytometry staining with anti-CD4, anti-CD8, anti-CD45RA, anti-CD45RO, anti-CD62L, anti-CCR7, anti-CD25 and anti-CD69 antibodies.

### Example 7

### TREM2 Cleavage

To confirm that the mutant TREM2 exodomain in the chimeric constructs of the invention is resistant to cleavage by the ADAM sheddases, Jurkat cells were transduced with four chimeric constructs of the invention comprising the mutant TREM2 exodomain, namely SEQ ID NOs: 57, 58, 59 and 60, and with a chimeric construct of the invention comprising the wild-type TREM2 exodomain, namely SEQ ID NO: 69.

The Jurkat cells were kept in culture and stained every 3 to 4 days with a TREM2 antibody, for up to 39 days, to confirm stable expression of the constructs in the cell membrane. Cell culture media was harvested the same day as staining. Harvested media was either frozen for later analysis by ELISA or protein was extracted for analysis by Western blot. A TREM2 antibody was used to detect the cleaved extracellular portion of TREM2, which is at around 28 kDa in the Western blot in FIGS. 11a and 11b. Cell lysates from Jurkat cells transduced with a chimeric construct of the invention (SEQ ID NO: 61) were used as a positive control for the Western blots to prove specificity of the TREM2 antibody and media from untransduced Jurkat cells (UTD—Jurkat media) was used as a negative control.

The Western blots in FIGS. 11a and 11b show that the constructs comprising the mutant TREM2 exodomain (SEQ ID NOs: 57-60) were resistant to cleavage by the ADAM sheddases, as less cleaved TREM2 was detected in media from cells transduced with these constructs compared to media from cells transduced with the construct comprising the wild-type TREM2 exodomain (SEQ ID NO: 69). This was the same at two different time points: day 11 (FIG. 11a) and day 14 (FIG. 11b). Similarly, it can be seen in the ELISA in FIG. 11c that there was less cleaved TREM2 in the media of cells transduced with constructs comprising the mutant TREM2 exodomain (SEQ ID NOs: 57-60) compared to cells comprising the wild-type TREM2 exodomain (SEQ ID NO: 69), at two different time points: day 24 and day 31.

1. A chimeric receptor comprising:
(a) an exodomain comprising the ligand binding domain of TREM2 or a functional variant thereof;
(b) a transmembrane domain; and
(c) an endodomain comprising an intracellular signalling domain.

2. The chimeric receptor of claim 1, wherein said exodomain is resistant to cleavage by a sheddase.

3. The chimeric receptor of claim 1 or claim 2, wherein the chimeric receptor comprises (a)-(c) in a single polypeptide chain.

4. The chimeric receptor of claim 1, 2 or 3, wherein the chimeric receptor comprises two or more polypeptide chains, wherein at least one of the polypeptide chains comprises linked domains from two or more proteins, optionally wherein the exodomain and endodomain are in different polypeptide chains.

5. The chimeric receptor of any one of claims 2 to 4, wherein the sheddase is a member of the ADAM (a disintegrin and metalloproteinase) protein family or is a member of the metalloproteinases, such as meprin β.

6. The chimeric receptor of any one of claims 2 to 5, wherein the sheddase is ADAM10 and/or ADAM 17.

7. The chimeric receptor of any one of claims 1 to 6, wherein the exodomain comprises:
(i) a functional variant of an amino acid sequence as set forth in SEQ ID NO: 3; or
(ii) a functional variant of an amino acid sequence as set forth in SEQ ID NO: 4,
wherein the amino acid at the position equivalent to position 78 of SEQ ID NO: 4 is a basic amino acid, preferably lysine or arginine.

8. The chimeric receptor of any one of claims 1 to 7, wherein the exodomain comprises or consists of an amino acid sequence as set forth in SEQ ID NO: 5 or 6 or a functional variant thereof,
wherein the amino acid at the position equivalent to position 78 of SEQ ID NO: 6 is a basic amino acid, preferably lysine or arginine.

9. The chimeric receptor of any one of claims 1 to 7, wherein the exodomain comprises or consists of:
(i) an amino acid sequence as set forth in SEQ ID NO: 7 or 8 or a functional variant thereof,
wherein
(a) the amino acid at the position equivalent to position 78 of SEQ ID NO: 8 is a basic amino acid, preferably lysine or arginine;
(b) the amino acids at positions equivalent to positions 139-140 of SEQ ID NO: 7 or 8 are:
(1) not histidine and/or serine, respectively; and/or
(2) modified to make the exodomain resistant to cleavage by the sheddase; and optionally
(c) the amino acids at the position equivalent to positions 118-119 of SEQ ID NO 7 or 8 are:
(1) not arginine and/or aspartic acid, respectively; and/or
(2) modified to make the exodomain resistant to cleavage by a sheddase.

10. The chimeric receptor of any one of claims 1 to 9, wherein the chimeric receptor comprises a hinge domain between the ligand binding domain of TREM2 and the transmembrane domain.

11. The chimeric receptor of claim 10, wherein the hinge domain is, or is derived from, the hinge region or stalk domain of human CD8α, CD4, CD28, CD7 or TREM2.

12. The chimeric receptor of any one of claims 1 to 11, wherein the chimeric receptor comprises a signal sequence upstream of the ligand binding domain of TREM2.

13. The chimeric receptor of claim 12, wherein the signal sequence is a CD8a signal sequence.

14. The chimeric receptor of any one of claims 1 to 13, wherein the chimeric receptor comprises one or more co-stimulatory signalling domains.

15. The chimeric receptor of claim 14, wherein the one or more co-stimulatory signalling domains is from a protein selected from CD27, CD28, 4-IBB (CD137), OX40 (CD134), CD30, CD40, ICOS (CD278), LFA-1, CD2, CD7, LIGHT, NKD2C, B7-H2 and a ligand that specifically binds CD83.

16. The chimeric receptor of any one of claims 1 to 15, wherein the transmembrane domain is from a protein selected from a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CDS, CD22, CD79a, CD79b, CD66d, TNF-alpha, NF-kappaB, a TLR (toll-like receptor), TLRS, Myd88, lymphocyte receptor chain, IL-2 receptor, IgE, IgG, CD16α, FcγRIII, FcγRII, CD28, 4-1BB, CD4, CD8, e.g. CD8α, NKG2D (CD314) and TREM2.

17. The chimeric receptor of any one of claims 1 to 16, wherein the intracellular signalling domain is from a protein selected from a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CDS, CD22, CD79a, CD79b, CD66d, TNF-alpha, NF-KappaB, a TLR (toll-like receptor), TLRS, Myd88, TOR/CD3 complex, lymphocyte receptor chain, IL-2 receptor, IgE, IgG, CD16α, FcγRIII, FcγCD28, 4-1BB, and any combination thereof.

18. The chimeric receptor of any one of claims 1 to 17, wherein the chimeric receptor comprises a signal peptide from CD8a; a hinge domain, transmembrane domain, co-stimulatory domain from CD28; and a CD3ζ intracellular signalling domain.

19. The chimeric receptor of any one of claims 1 to 18, wherein the chimeric receptor comprises an amino acid sequence of any one of SEQ ID NO. 32, 33 or 40 to 56, or an amino acid having at least 90% (e.g. at least 95%) sequence identity thereto.

20. One or a plurality of nucleic acid molecules encoding a chimeric receptor of any one of claims 1 to 19.

21. A vector comprising the one or plurality of nucleic acid molecules of claim 20, optionally wherein the vector encodes a chimeric receptor having an amino acid sequence of any one of SEQ ID Nos 32, 33 or 40 to 56, or a sequence having at least 90% identity thereto.

22. A cell, preferably an immune cell, comprising the one or more nucleic acid molecules of claim 20 or vector of claim 21 and/or expressing the chimeric receptor of any one of claims 1 to 19.

23. The cell of claim 22, wherein the cell is an NK cell, a dendritic cell, a NKT cell, a MDSC, a neutrophil, a macrophage or a T cell, such as a cytotoxic T lymphocyte (CTL), helper T cell or a Treg cell.

24. A cell population comprising the cell of claim 22 or 23.

25. A pharmaceutical composition comprising the cell of claim 22 or 23 or the cell population of claim 24.

26. A cell, cell population or pharmaceutical composition of any preceding claim for use in therapy.

27. A cell, cell population or pharmaceutical composition of any preceding claim for use in preventing, reducing risk of, or treating a neurological disease, disorder, or injury or liver disease in an individual in need thereof.

28. A cell, cell population or pharmaceutical composition for use of claim 27, wherein the neurological disease, disorder, or injury is selected from amyotrophic lateral sclerosis (ALS), dementia, frontotemporal dementia, Alzheimer's disease, vascular dementia, mixed dementia, Creutzfeldt-Jakob disease, Chronic Inflammatory Demyelinating Polyneuropathy (CIDP), Huntington's disease, Taupathy disease, Nasu-Hakola disease, central nervous system lupus, Parkinson's disease, dementia with Lewy bodies, Multiple System Atrophy (Shy-Drager syndrome), progressive supranuclear palsy, cortical basal ganglionic degeneration, acute disseminated encephalomyelitis, seizures, spinal cord injury, traumatic brain injury (e.g. ischemia and traumatic brain injury), depression, autism spectrum disorder and multiple sclerosis.

29. A cell, cell population or pharmaceutical composition for use of claim 27, wherein the neurological disease is amyotrophic lateral sclerosis (ALS) and the cell is a regulatory T cell (Treg) and the cell population is a regulatory T cell (Treg) population.

30. A cell, cell population or pharmaceutical composition for use of claim 27, wherein the liver disease is selected from fascioliasis, hepatitis (e.g. viral hepatitis, alcoholic hepatitis or autoimmune hepatitis), alcoholic liver disease, fatty liver disease (hepatic steatosis and/or steatohepatitis), hemochromatosis, Gilberts syndrome, cirrhosis, primary biliary cirrhosis and primary sclerosing cholangitis.

31. A cell, cell population or pharmaceutical composition of any preceding claim for use in preventing, reducing risk of, or treating fibrosis or atherosclerosis in an individual in need thereof.

32. The cell, cell population or pharmaceutical composition for use of claim 31, wherein said fibrosis is fibrosis of the kidneys, lungs, liver, brain, intestines, heart or a combination thereof.
